SEQUENCE LISTING (37 sequences)

SEQ ID NO: 1 - DNA, 5501 bp, synthetic construct
  source 1..5501: mol_type = other DNA
aagcttgagctcgcatgcttcggcgagtttagttgcgtcgaactcatcaggtgtttgggaataagtgaaaagccattttttacatcggagtcggtatctgtgatcactagttagttagttgggaatagcttcactttttggcaattgagttaagtaagggtcattgcaccatcatgggggagggacctccgggggggccccatgtggggctgtacccgcgtcagaagcccaagctctggaataaaaaaaataaaaaatacgtactggttgcgttgtcgtttttgtggctgaggtgtttctgagtcacatgattcggaagcgcgtttggacattgtggctgaagagtggctttgcggaggtcaagaaatctaagaaagagaggatttaagattcttctctttcttagatttgtgaccgtgacccaagtttgatttatatactagttatgtgttgactaaggtacagagaggggtagtgatctccacaataatggcgttagcgtccatccatttccaatcaatatcagtgcaggtacggcgtggatcttcattggctatgtagatgcacggcttaccccacatgacccgctgttttcctttgtacttgtcggtagctacgaattctcgttggccgaaccattgtttgtaatttggaaaagaggagaacccaccaatcaagtcatcaaacaccgcgtagtttacgttagggtgaaagtcagagagcatgaacatgcctgggaagtaggcgtgattggctagacttcgcgcccatagagttttaccgaggcgggaagcgccgaataaaatgagacttatgggtcggtatgatgtgttgttcgtgacgtaattgtcaacccagtctttgagttcgggatagtcagagtaattgacagtaatttgtggagattcgtatgtgctctgtggtggaccgaatttccattcagcaaatgacttgatgttgttccagcatttgatggattggttaggtagttcggtggcagccaagtgtaaaaattcttttgcggaagttgattggtcgatgatacgcaaccattctttatccatgttcttttggttcaaggatggttggtgtgggggtgtaccctcttcccattggatgtcgttatctttttttacgtaattgacgacagtgtgtggggttctggtgacaacttcgatgttggggtgaacaccacagaaatcgaagtcccgtgcgttgtttgaagtgtgaatagtttcaacttcccaatacacgtgatggtggaatccgccatctttgtgttgttcttttgagatgacgagatagacaagaagtggacttttttctttgaacatttcggcgagtttagttgcgtcgaactcatcaggtgtttgggaataagtgaaaagccattttttacatcggagtcggtatctgtgatcactagttagttagttgggaatagcttcactttttggcaattgagttaagtaagggtcattgcaccatcatgggggagggacctccgggggggccccatgtggggctgtacccgcgtcagaagcccaagctctggaataaaaaaaataaaaaatacgtactggttgcgttgtcgtttttgtggctgaggtgtttctgagtcacatgattcggaagcgcgtttggacattgtggctgaagagtggctttgcggaggtcaagaaatctaagaaagagaggatttaagattcttctctttcttagatttgtgaccgtgacccaagtttgatttatatactaggcatgcgctagcggtaattttagtgtagcaaaattgagttggtgatagcttcatttttttggatccactttgtgacaattgagttgaagtaagggtcattgcaccatcatgggggagggacctccgggggggccccatgtggggctgtacccgcgtcagaagcccaagctccggaataaaaaaaaatcaaaagtagactgtcggttaaagttcggttgggtaggattagtcagcaaattttcaaccaatagcggaggtcaagaaatctaagaaagagaggatttaagattcttctctttcttagatttgtgaccgtgacccaggtttgattttgggtatataagggaggggtagccaccatttttgctagtctgtttttggacttaaaaaatttattttttaacacaaaacattattacgtcgacaaaaatggcttctacaaagaagaaatcatacaacaacaagaaggcttataaaaaaaaagaatggaagtcgaagaagacttgggacaagtctagttattacgacaattaccagtcgaagatgaatatttcgaatatgcagacgaagagggacaacatgatgtgtgtgacgtcacattgtggtgttccgaatgcggcgttactggagaattctgttgtgggtgaaattccagccaatatgggagttcattatattatgtggtctcctacgtatcgagaggcggtaccaccgaatcgagcggcacagttggatcggcaatccgcaaacacattttttactggttggaaggataatttgtcctatcaatttaagggacagattacagggattcacctgagggttgtgatatctacccgaagagaagtggagtccgcgcagccttttattgggccggggaatacgctgtgcagaaacttggcggttcgtgatatgtcggatgagacattggaccagtttttgtcgggtacccgggatgttgattggacgttggtgaatgtgatggacacgatgtttgatccggcggtgtgcaaggtgttgtttcggcagaggaagattttaggtgcagctgatgcgttgttgaagacggaggagttttatcaccgtatccgtcggcctatggtgtacggcgataggcaggatggtttggagtttgtgtctagtggttgggctggaagggagtcggagaacatatacgtcattgatatgtactctttgatttcggcagccccaccgttaggtaatttgttggatggagagggaaatattgttttggatgacaagaaacggcctattcccgtatatgcgaagttaaatattagtggaaatagtatagtgtattggagggagtagggtaattttagtgtagcaaaattgagttggtgatagcttcatttttttggatccactttgtgacaattgagttgaagtaagggtcattgcaccatcatgggggagggacctccgggggggccccatgtggggctgtacccgcgtcagaagcccaagctccggaataaaaaaaaatcaaaagtagactgtcggttaaagttcggttgggtaggattagtcagcaaattttcaaccaatagcggaggtcaagaaatctaagaaagagaggatttaagattcttctctttcttagatttgtgaccgtgacccaggtttgattttgggtatataagggaggggtagccaccatttttgctagtctgtttttggacttaaaaaatttattttttaacacaaaacattattacgtcgacaaaagctagcacgcgtccatggcaattgctttatattgtaaaaaatatttgtaactgtaaataattagttggtgatagcttcattttttttactccactttgtgacaattgagttaagtaagggtcattgcaccatcatgggggagggacctccgggggggccccatgtggggctgtacccgcgtcagaagcccaagctctttgttgagccgagcgcagcggtaatttggagtcacgtgaggtaaaataaaatgtggacttacgttcttggaattgatgattgagacattttgaaaaagtgttggagtggttggggtatttatggtcaaggacatgtttggtggtgtcattggttaatataggtactgtcggtagatagttgttgcggttgaagtataatgcgtggagcaccgaggtcagaacttttaggaaagacgatttaagattctctttcctaaaagtctgaccgtgactccctttggccttgacgacgttattggtggaggattggaatgttacccgcaatttcacgtgacatgtggaaatgtggtgacatgaagaattgtgggacggcacaattttaattgggtggaacacagcagggtaggattaggcagaatgaggcagatttaggcagcggaaatttatttttaaattggagcattgtctaaatctagaagtacatggtaccagtcatcatcgtttggttgatgatctggtatgtccacccaatcaggatcatttaaattatgtacaattgaattgttatgatttgtaaaaaaaaaagataggtaatcgcataacgtgtttttgttgaataaaataatacacgtgtttgcgtattgttgtaccatgtcatgtgggtcgggattgttgtggagatgacacgtgttagtgctggtatgaactcgcaggagtgtagtaccagtcgacaaaggcttggatgtgttggcggtgtcgttggagtagtgtagggtgagtttcctcgaaacgactaagaaacgccccttgatttggctcggagaaaagttgttgccagaaggggtcagatgtggctgagcaaagatctgcatccccgaactgagccacaagcacctctttagactggatgctctcgtataccgggcctgcatgtgtgtatattggcagaatagaaggcataactccgctgtaagcgagttgtaggcttcgttggaggggaatgtttccagtatcgatgtagatatcgagatcacagttcccattagttcttgtattgtaaccggcgatgaatctctttggattgatgtccatttggagttgagaagtgaaattatctgattgttttgggtcgacatctttatattgtaaaaaatatttgtaactgtaaataattagttggtgatagcttcattttttttactccactttgtgacaattgagttaagtaagggtcattgcaccatcatgggggagggacctccgggggggccccatgtggggctgtacccgcgtcagaagcccaagctctttgttgagccgagcgcagcggtaatttggagtcacgtgaggtaaaataaaatgtggacttacgttcttggaattgatgattgagacattttgaaaaagtgttggagtggttggggtatttatggtcaaggacatgtttggtggtgtcattggttaatataggtactgtcggtagatagttgttgcggttgaagtataatgcgtggagcaccgaggtcagaacttttaggaaagacgatttaagattctctttcctaaaagtctgaccgtgactccctttggccttgacgacgttattggtggaggattggaatgttacccgcaatttcacgtgacatgtggaaatgtggtgacatgaagaattgtgggacggcacaattttaattgggtggaacacagcagggtaggattaggcagaatgaggcagatttaggcagcggaaatttatttacgcgtccatggcaattggagctcctgcaggcggccgc

SEQ ID NO: 2 - DNA, 5369 bp, synthetic construct
  source 1..5369: mol_type = other DNA
aagcttgagctcgcatgcttcggcgagtttagttgcgtcgaactcatcaggtgtttgggaataagtgaaaagccattttttacatcggagtcggtatctgtgatcactagttagttagttgggaatagcttcactttttggcaattgagttaagtaagggtcattgcaccatcatgggggagggacctccgggggggccccatgtggggctgtacccgcgtcagaagcccaagctctggaataaaaaaaataaaaaatacgtactggttgcgttgtcgtttttgtggctgaggtgtttctgagtcacatgattcggaagcgcgtttggacattgtggctgaagagtggctttgcggaggtcaagaaatctaagaaagagaggatttaagattcttctctttcttagatttgtgaccgtgacccaagtttgatttatatactagttatgtgttgactaaggtacagagaggggtagtgatctccacaataatggcgttagcgtccatccatttccaatcaatatcagtgcaggtacggcgtggatcttcattggctatgtagatgcacggcttaccccacatgacccgctgttttcctttgtacttgtcggtagctacgaattctcgttggccgaaccattgtttgtaatttggaaaagaggagaacccaccaatcaagtcatcaaacaccgcgtagtttacgttagggtgaaagtcagagagcatgaacatgcctgggaagtaggcgtgattggctagacttcgcgcccatagagttttaccgaggcgggaagcgccgaataaaatgagacttatgggtcggtatgatgtgttgttcgtgacgtaattgtcaacccagtctttgagttcgggatagtcagagtaattgacagtaatttgtggagattcgtatgtgctctgtggtggaccgaatttccattcagcaaatgacttgatgttgttccagcatttgatggattggttaggtagttcggtggcagccaagtgtaaaaattcttttgcggaagttgattggtcgatgatacgcaaccattctttatccatgttcttttggttcaaggatggttggtgtgggggtgtaccctcttcccattggatgtcgttatctttttttacgtaattgacgacagtgtgtggggttctggtgacaacttcgatgttggggtgaacaccacagaaatcgaagtcccgtgcgttgtttgaagtgtgaatagtttcaacttcccaatacacgtgatggtggaatccgccatctttgtgttgttcttttgagatgacgagatagacaagaagtggacttttttctttgaacatttcggcgagtttagttgcgtcgaactcatcaggtgtttgggaataagtgaaaagccattttttacatcggagtcggtatctgtgatcactagttagttagttgggaatagcttcactttttggcaattgagttaagtaagggtcattgcaccatcatgggggagggacctccgggggggccccatgtggggctgtacccgcgtcagaagcccaagctctggaataaaaaaaataaaaaatacgtactggttgcgttgtcgtttttgtggctgaggtgtttctgagtcacatgattcggaagcgcgtttggacattgtggctgaagagtggctttgcggaggtcaagaaatctaagaaagagaggatttaagattcttctctttcttagatttgtgaccgtgacccaagtttgatttatatactaggcatgcgctagcggtaattttagtgtagcaaaattgagttggtgatagcttcatttttttggatccactttgtgacaattgagttgaagtaagggtcattgcaccatcatgggggagggacctccgggggggccccatgtggggctgtacccgcgtcagaagcccaagctccggaataaaaaaaaatcaaaagtagactgtcggttaaagttcggttgggtaggattagtcagcaaattttcaaccaatagcggaggtcaagaaatctaagaaagagaggatttaagattcttctctttcttagatttgtgaccgtgacccaggtttgattttgggtatataagggaggggtagccaccatttttgctagtctgtttttggacttaaaaaatttattttttaacacaaaacattattacgtcgacaaaaatggcttctacaaagaagaaatcatacaacaacaagaaggcttataaaaaaaaagaatggaagtcgaagaagacttgggacaagtctagttattacgacaattaccagtcgaagatgaatatttcgaatatgcagacgaagagggacaacatgatgtgtgtgacgtcacattgtggtgttccgaatgcggcgttactggagaattctgttgtgggtgaaattccagccaatatgggagttcattatattatgtggtctcctacgtatcgagaggcggtaccaccgaatcgagcggcacagttggatcggcaatccgcaaacacattttttactggttggaaggataatttgtcctatcaatttaagggacagattacagggattcacctgagggttgtgatatctacccgaagagaagtggagtccgcgcagccttttattgggccggggaatacgctgtgcagaaacttggcggttcgtgatatgtcggatgagacattggaccagtttttgtcgggtacccgggatgttgattggacgttggtgaatgtgatggacacgatgtttgatccggcggtgtgcaaggtgttgtttcggcagaggaagattttaggtgcagctgatgcgttgttgaagacggaggagttttatcaccgtatccgtcggcctatggtgtacggcgataggcaggatggtttggagtttgtgtctagtggttgggctggaagggagtcggagaacatatacgtcattgatatgtactctttgatttcggcagccccaccgttaggtaatttgttggatggagagggaaatattgttttggatgacaagaaacggcctattcccgtatatgcgaagttaaatattagtggaaatagtatagtgtattggagggagtagggtaattttagtgtagcaaaattgagttggtgatagcttcatttttttggatccactttgtgacaattgagttgaagtaagggtcattgcaccatcatgggggagggacctccgggggggccccatgtggggctgtacccgcgtcagaagcccaagctccggaataaaaaaaaatcaaaagtagactgtcggttaaagttcggttgggtaggattagtcagcaaattttcaaccaatagcggaggtcaagaaatctaagaaagagaggatttaagattcttctctttcttagatttgtgaccgtgacccaggtttgattttgggtatataagggaggggtagccaccatttttgctagtctgtttttggacttaaaaaatttattttttaacacaaaacattattacgtcgacaaaagctagcacgcgtccatggcaattgctttatattgtaaaaaatatttgtaactgtaaataattagttggtgatagcttcattttttttactccactttgtgacaattgagttaagtaagggtcattgcaccatcatgggggagggacctccgggggggccccatgtggggctgtacccgcgtcagaagcccaagctctttgttgagccgagcgcagcggtaatttggagtcacgtgaggtaaaataaaatgtggacttacgttcttggaattgatgattgagacattttgaaaaagtgttggagtggttggggtatttatggtcaaggacatgtttggtggtgtcattggttaatataggtactgtcggtagatagttgttgcggttgaagtataatgcgtggagcaccgaggtcagaacttttaggaaagacgatttaagattctctttcctaaaagtctgaccgtgactccctttggccttgacgacgttattggtggaggattggaatgttacccgcaatttcacgtgacatgtggaaatgtggtgacatgaagaattgtgggacggcacaattttaattgggtggaacacagcagggtaggattaggcagaatgaggcagatttaggcagcggaaatttatttttaaattggagcattgtctaaatctagaagtacatggtaccagtcatcatcgtttggttgatgatctggtatgtccacccaatcatgcatcctaggaccggtacacgtgttagtgctggtatgaactcgcaggagtgtagtaccagtcgacaaaggcttggatgtgttggcggtgtcgttggagtagtgtagggtgagtttcctcgaaacgactaagaaacgccccttgatttggctcggagaaaagttgttgccagaaggggtcagatgtggctgagcaaagatctgcatccccgaactgagccacaagcacctctttagactggatgctctcgtataccgggcctgcatgtgtgtatattggcagaatagaaggcataactccgctgtaagcgagttgtaggcttcgttggaggggaatgtttccagtatcgatgtagatatcgagatcacagttcccattagttcttgtattgtaaccggcgatgaatctctttggattgatgtccatttggagttgagaagtgaaattatctgattgttttgggtcgacatctttatattgtaaaaaatatttgtaactgtaaataattagttggtgatagcttcattttttttactccactttgtgacaattgagttaagtaagggtcattgcaccatcatgggggagggacctccgggggggccccatgtggggctgtacccgcgtcagaagcccaagctctttgttgagccgagcgcagcggtaatttggagtcacgtgaggtaaaataaaatgtggacttacgttcttggaattgatgattgagacattttgaaaaagtgttggagtggttggggtatttatggtcaaggacatgtttggtggtgtcattggttaatataggtactgtcggtagatagttgttgcggttgaagtataatgcgtggagcaccgaggtcagaacttttaggaaagacgatttaagattctctttcctaaaagtctgaccgtgactccctttggccttgacgacgttattggtggaggattggaatgttacccgcaatttcacgtgacatgtggaaatgtggtgacatgaagaattgtgggacggcacaattttaattgggtggaacacagcagggtaggattaggcagaatgaggcagatttaggcagcggaaatttatttacgcgtccatggcaattggagctcctgcaggcggccgc

SEQ ID NO: 3 - DNA, 150 bp, synthetic construct
  source 1..150: mol_type = other DNA
gatttgtactctgttcccaagcgtcatctttctcagcgcagcacttctataatttagcggcctcaccttctgtaacaccaacaccaagtgatttacaaacaccaccaaaatggctttcaagatacagctcgacaccctcggccagctacc

SEQ ID NO: 4 - DNA, 150 bp, synthetic construct
  source 1..150: mol_type = other DNA
accatctgcttgctcctcgcctacaagatcaagtaccccgaaaacttcttcatccttcgaggtaaccacgagtgtgcctccatcaaccgtatttatggattctacgacgagtgcaagcgtcgctataacatcaagttgtggaagactttc

SEQ ID NO: 5 - DNA, 1741 bp, synthetic construct
  source 1..1741: mol_type = other DNA
ttcggcgagtttagttgcgtcgaactcatcaggtgtttgggaataagtgaaaagccattttttacatcggagtcggtatctgtgatcactagttagttagttgggaatagcttcactttttggcaattgagttaagtaagggtcattgcaccatcatgggggagggacctccgggggggccccatgtggggctgtacccgcgtcagaagcccaagctctggaataaaaaaaataaaaaatacgtactggttgcgttgtcgtttttgtggctgaggtgtttctgagtcacatgattcggaagcgcgtttggacattgtggctgaagagtggctttgcggaggtcaagaaatctaagaaagagaggatttaagattcttctctttcttagatttgtgaccgtgacccaagtttgatttatatactagttatgtgttgactaaggtacagagaggggtagtgatctccacaataatggcgttagcgtccatccatttccaatcaatatcagtgcaggtacggcgtggatcttcattggctatgtagatgcacggcttaccccacatgacccgctgttttcctttgtacttgtcggtagctacgaattctcgttggccgaaccattgtttgtaatttggaaaagaggagaacccaccaatcaagtcatcaaacaccgcgtagtttacgttagggtgaaagtcagagagcatgaacatgcctgggaagtaggcgtgattggctagacttcgcgcccatagagttttaccgaggcgggaagcgccgaataaaatgagacttatgggtcggtatgatgtgttgttcgtgacgtaattgtcaacccagtctttgagttcgggatagtcagagtaattgacagtaatttgtggagattcgtatgtgctctgtggtggaccgaatttccattcagcaaatgacttgatgttgttccagcatttgatggattggttaggtagttcggtggcagccaagtgtaaaaattcttttgcggaagttgattggtcgatgatacgcaaccattctttatccatgttcttttggttcaaggatggttggtgtgggggtgtaccctcttcccattggatgtcgttatctttttttacgtaattgacgacagtgtgtggggttctggtgacaacttcgatgttggggtgaacaccacagaaatcgaagtcccgtgcgttgtttgaagtgtgaatagtttcaacttcccaatacacgtgatggtggaatccgccatctttgtgttgttcttttgagatgacgagatagacaagaagtggacttttttctttgaacatttcggcgagtttagttgcgtcgaactcatcaggtgtttgggaataagtgaaaagccattttttacatcggagtcggtatctgtgatcactagttagttagttgggaatagcttcactttttggcaattgagttaagtaagggtcattgcaccatcatgggggagggacctccgggggggccccatgtggggctgtacccgcgtcagaagcccaagctctggaataaaaaaaataaaaaatacgtactggttgcgttgtcgtttttgtggctgaggtgtttctgagtcacatgattcggaagcgcgtttggacattgtggctgaagagtggctttgcggaggtcaagaaatctaagaaagagaggatttaagattcttctctttcttagatttgtgaccgtgacccaagtttgatttatatactag

SEQ ID NO: 6 - DNA, 1737 bp, synthetic construct
  source 1..1737: mol_type = other DNA
ggtaattttagtgtagcaaaattgagttggtgatagcttcatttttttggatccactttgtgacaattgagttgaagtaagggtcattgcaccatcatgggggagggacctccgggggggccccatgtggggctgtacccgcgtcagaagcccaagctccggaataaaaaaaaatcaaaagtagactgtcggttaaagttcggttgggtaggattagtcagcaaattttcaaccaatagcggaggtcaagaaatctaagaaagagaggatttaagattcttctctttcttagatttgtgaccgtgacccaggtttgattttgggtatataagggaggggtagccaccatttttgctagtctgtttttggacttaaaaaatttattttttaacacaaaacattattacgtcgacaaaaatggcttctacaaagaagaaatcatacaacaacaagaaggcttataaaaaaaaagaatggaagtcgaagaagacttgggacaagtctagttattacgacaattaccagtcgaagatgaatatttcgaatatgcagacgaagagggacaacatgatgtgtgtgacgtcacattgtggtgttccgaatgcggcgttactggagaattctgttgtgggtgaaattccagccaatatgggagttcattatattatgtggtctcctacgtatcgagaggcggtaccaccgaatcgagcggcacagttggatcggcaatccgcaaacacattttttactggttggaaggataatttgtcctatcaatttaagggacagattacagggattcacctgagggttgtgatatctacccgaagagaagtggagtccgcgcagccttttattgggccggggaatacgctgtgcagaaacttggcggttcgtgatatgtcggatgagacattggaccagtttttgtcgggtacccgggatgttgattggacgttggtgaatgtgatggacacgatgtttgatccggcggtgtgcaaggtgttgtttcggcagaggaagattttaggtgcagctgatgcgttgttgaagacggaggagttttatcaccgtatccgtcggcctatggtgtacggcgataggcaggatggtttggagtttgtgtctagtggttgggctggaagggagtcggagaacatatacgtcattgatatgtactctttgatttcggcagccccaccgttaggtaatttgttggatggagagggaaatattgttttggatgacaagaaacggcctattcccgtatatgcgaagttaaatattagtggaaatagtatagtgtattggagggagtagggtaattttagtgtagcaaaattgagttggtgatagcttcatttttttggatccactttgtgacaattgagttgaagtaagggtcattgcaccatcatgggggagggacctccgggggggccccatgtggggctgtacccgcgtcagaagcccaagctccggaataaaaaaaaatcaaaagtagactgtcggttaaagttcggttgggtaggattagtcagcaaattttcaaccaatagcggaggtcaagaaatctaagaaagagaggatttaagattcttctctttcttagatttgtgaccgtgacccaggtttgattttgggtatataagggaggggtagccaccatttttgctagtctgtttttggacttaaaaaatttattttttaacacaaaacattattacgtcgacaaaa

SEQ ID NO: 7 - DNA, 1931 bp, synthetic construct
  source 1..1931: mol_type = other DNA
ctttatattgtaaaaaatatttgtaactgtaaataattagttggtgatagcttcattttttttactccactttgtgacaattgagttaagtaagggtcattgcaccatcatgggggagggacctccgggggggccccatgtggggctgtacccgcgtcagaagcccaagctctttgttgagccgagcgcagcggtaatttggagtcacgtgaggtaaaataaaatgtggacttacgttcttggaattgatgattgagacattttgaaaaagtgttggagtggttggggtatttatggtcaaggacatgtttggtggtgtcattggttaatataggtactgtcggtagatagttgttgcggttgaagtataatgcgtggagcaccgaggtcagaacttttaggaaagacgatttaagattctctttcctaaaagtctgaccgtgactccctttggccttgacgacgttattggtggaggattggaatgttacccgcaatttcacgtgacatgtggaaatgtggtgacatgaagaattgtgggacggcacaattttaattgggtggaacacagcagggtaggattaggcagaatgaggcagatttaggcagcggaaatttatttttaaattggagcattgtctaaatctagaagtacatggtaccagtcatcatcgtttggttgatgatctggtatgtccacccaatcaggatcatttaaattatgtacaattgaattgttatgatttgtaaaaaaaaaagataggtaatcgcataacgtgtttttgttgaataaaataatacacgtgtttgcgtattgttgtaccatgtcatgtgggtcgggattgttgtggagatgacacgtgttagtgctggtatgaactcgcaggagtgtagtaccagtcgacaaaggcttggatgtgttggcggtgtcgttggagtagtgtagggtgagtttcctcgaaacgactaagaaacgccccttgatttggctcggagaaaagttgttgccagaaggggtcagatgtggctgagcaaagatctgcatccccgaactgagccacaagcacctctttagactggatgctctcgtataccgggcctgcatgtgtgtatattggcagaatagaaggcataactccgctgtaagcgagttgtaggcttcgttggaggggaatgtttccagtatcgatgtagatatcgagatcacagttcccattagttcttgtattgtaaccggcgatgaatctctttggattgatgtccatttggagttgagaagtgaaattatctgattgttttgggtcgacatctttatattgtaaaaaatatttgtaactgtaaataattagttggtgatagcttcattttttttactccactttgtgacaattgagttaagtaagggtcattgcaccatcatgggggagggacctccgggggggccccatgtggggctgtacccgcgtcagaagcccaagctctttgttgagccgagcgcagcggtaatttggagtcacgtgaggtaaaataaaatgtggacttacgttcttggaattgatgattgagacattttgaaaaagtgttggagtggttggggtatttatggtcaaggacatgtttggtggtgtcattggttaatataggtactgtcggtagatagttgttgcggttgaagtataatgcgtggagcaccgaggtcagaacttttaggaaagacgatttaagattctctttcctaaaagtctgaccgtgactccctttggccttgacgacgttattggtggaggattggaatgttacccgcaatttcacgtgacatgtggaaatgtggtgacatgaagaattgtgggacggcacaattttaattgggtggaacacagcagggtaggattaggcagaatgaggcagatttaggcagcggaaatttattt

SEQ ID NO: 8 - DNA, 39 bp, synthetic construct
  source 1..39: mol_type = other DNA
aggatgcattacaactcgcttacagcggagttatgcctt

SEQ ID NO: 9 - DNA, 39 bp, synthetic construct
  source 1..39: mol_type = other DNA
aggaccggtcatctttatattgtaaaaaatatttgtaac

SEQ ID NO: 10 - DNA, 39 bp, synthetic construct
  source 1..39: mol_type = other DNA
aggatgcataacaacttttctccgagccaaatcaagggg

SEQ ID NO: 11 - DNA, 39 bp, synthetic construct
  source 1..39: mol_type = other DNA
aggaccggtggcttcgttggaggggaatgtttccagtat

SEQ ID NO: 12 - DNA, 39 bp, synthetic construct
  source 1..39: mol_type = other DNA
aggatgcatcatctccacaacaatcccgacccacatgac

SEQ ID NO: 13 - DNA, 39 bp, synthetic construct
  source 1..39: mol_type = other DNA
aggaccggtgccagaaggggtcagatgtggctgagcaaa

SEQ ID NO: 14 - DNA, 39 bp, synthetic construct
  source 1..39: mol_type = other DNA
aggatgcatgattgggtggacataccagatcatcaacca

SEQ ID NO: 15 - DNA, 39 bp, synthetic construct
  source 1..39: mol_type = other DNA
aggaccggtacacgtgttagtgctggtatgaactcgcag

SEQ ID NO: 16 - DNA, 39 bp, synthetic construct
  source 1..39: mol_type = other DNA
aggatgcattaaaaataaatttccgctgcctaaatctgc

SEQ ID NO: 17 - DNA, 39 bp, synthetic construct
  source 1..39: mol_type = other DNA
aggaccggtaggatcatttaaattatgtacaattgaatt

SEQ ID NO: 18 - DNA, 24 bp, synthetic construct
  source 1..24: mol_type = other DNA
cccaccggtcagcacgacttcttc

SEQ ID NO: 19 - DNA, 25 bp, synthetic construct
  source 1..25: mol_type = other DNA
ccaatgcatgccgtcgtccttgaag

SEQ ID NO: 20 - DNA, 25 bp, synthetic construct
  source 1..25: mol_type = other DNA
cccaccggtgccgtcgtccttgaag

SEQ ID NO: 21 - DNA, 24 bp, synthetic construct
  source 1..24: mol_type = other DNA
ccaatgcatcagcacgacttcttc

SEQ ID NO: 22 - DNA, 27 bp, synthetic construct
  source 1..27: mol_type = other DNA
cccaccggtatggtgagcaagggcgag

SEQ ID NO: 23 - DNA, 25 bp, synthetic construct
  source 1..25: mol_type = other DNA
ccaatgcatggtgcagatgaacttc

SEQ ID NO: 24 - DNA, 25 bp, synthetic construct
  source 1..25: mol_type = other DNA
cccaccggtggtgcagatgaacttc

SEQ ID NO: 25 - DNA, 27 bp, synthetic construct
  source 1..27: mol_type = other DNA
ccaatgcatatggtgagcaagggcgag

SEQ ID NO: 26 - DNA, 27 bp, synthetic construct
  source 1..27: mol_type = other DNA
cccaccggtcagcacgacttcttcaag

SEQ ID NO: 27 - DNA, 25 bp, synthetic construct
  source 1..25: mol_type = other DNA
ccaatgcatggcgagctgcacgctg

SEQ ID NO: 28 - DNA, 26 bp, synthetic construct
  source 1..26: mol_type = other DNA
cccaccggtggcgagctgcacgctgc

SEQ ID NO: 29 - DNA, 27 bp, synthetic construct
  source 1..27: mol_type = other DNA
ccaatgcatcagcacgacttcttcaag

SEQ ID NO: 30 - DNA, 27 bp, synthetic construct
  source 1..27: mol_type = other DNA
cccaccggtatggtgagcaagggcgag

SEQ ID NO: 31 - DNA, 25 bp, synthetic construct
  source 1..25: mol_type = other DNA
ccagttgtggctgttgtagttgtac

SEQ ID NO: 32 - DNA, 25 bp, synthetic construct
  source 1..25: mol_type = other DNA
cccaccggtgttgtggctgttgtag

SEQ ID NO: 33 - DNA, 27 bp, synthetic construct
  source 1..27: mol_type = other DNA
ccaatgcatatggtgagcaagggcgag

SEQ ID NO: 34 - DNA, 40 bp, synthetic construct
  source 1..40: mol_type = other DNA
cagcactaacacgtgtaccggtgatttgtactctgttccc

SEQ ID NO: 35 - DNA, 42 bp, synthetic construct
  source 1..42: mol_type = other DNA
gtatgtccacccaatcatgcatggtagctggccgagggtgtc

SEQ ID NO: 36 - DNA, 43 bp, synthetic construct
  source 1..43: mol_type = other DNA
cagcactaacacgtgtaccggtaccatctgcttgctcctcgcc

SEQ ID NO: 37 - DNA, 41 bp, synthetic construct
  source 1..41: mol_type = other DNA
gtatgtccacccaatcatgcatgaaagtcttccacaacttg

(19) United States
(12) Patent Application Publication
(10) Pub. No.: US 2024/0352471 A1
(43) Pub. Date: Oct. 24, 2024

(54) MYCOVIRUS-INDUCED GENE SILENCING VECTOR, CONSTRUCTION METHOD AND APPLICATION THEREOF

(71) Applicants: Institute of Plant Protection(IPP), Chinese Academy of Agricultural Sciences(CAAS), Beijing (CN); Beijing Zhongbao Green Agricultural Science and Technology Group Co., Ltd, Beijing (CN)

(72) Inventors: Lihua GUO, Beijing (CN); Chang CHEN, Beijing (CN); Lihang ZHANG, Beijing (CN); Shuangchao WANG, Beijing (CN); Shaojian RUAN, Beijing (CN); Jun XU, Beijing (CN); Yijun ZHAO, Beijing (CN)

(21) Appl. No.: 18/529,537

(22) Filed: Dec. 5, 2023

Related U.S. Application Data
(63) Continuation of application No. PCT/CN2023/097344, filed on May 31, 2023.

(30) Foreign Application Priority Data
Apr. 23, 2023 (CN) 202310441503.1

(51) Int. Cl. C12N 15/80; C12N 15/10; C12R 1/77

(52) U.S. Cl. CPC C12N 15/80; C12N 9/1029; C12N 9/16; C12N 15/102; C12N 2750/00022; C12N 2750/00031; C12N 2750/00043; C12R 2001/77

(57) ABSTRACT

A mycovirus-induced gene silencing vector and a construction method and an application thereof are provided. A nucleotide sequence of the mycovirus-induced gene silencing vector is shown in SEQ ID NO: 2, and construction method for the mycovirus-induced gene silencing vector includes: (1) connecting three single-stranded circular DNA molecules DNA-A, DNA-B and DNA-C of the mycovirus FgGMTV1/HB58 in series and introducing them into a same vector to construct a recombinant vector; and (2) carrying out a deletion mutation on a coding protein p26 of the DNA-C molecule in the recombinant vector to obtain the mycovirus-induced gene silencing vector.

### CROSS-REFERENCE TO RELATED APPLICATIONS

This application is a continuation of PCT/CN2023/097344, filed on May 31, 2023, and claims priority of Chinese Patent Application No. 202310441503.1, filed on Apr. 23, 2023, the entire contents of which are incorporated herein by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: sequence_347095_12676
Creation date: Nov. 2, 2023
Byte size: 50,704

### TECHNICAL FIELD

The disclosure relates to the field of genetic engineering, and in particular to a mycovirus-induced gene silencing vector and a construction method and application thereof.

### BACKGROUND

Virus-induced gene silencing (VIGS) is a technique to study the function of plant genes by RNA-mediated plant antiviral mechanism. The principle is to insert the target gene fragment into the virus vector and infect the host plant. While the immune system of the plant recognizes the virus and the target gene, it degrades the endogenous target gene mRNA, causing the plant to have a phenotype of loss of function or decreased expression level of the target gene. When VIGS is initiated, a large number of double-stranded RNA (dsRNA) are synthesized by RNA-mediated RNA polymerase. In cells, dsRNA is cleaved into 19-24 nt silencing RNA (siRNA) by Dicer analogues of RNaseIII family-specific endonucleases. SiRNA binds to a specific protein in the form of a single strand to form an RNA-induced silencing complex (RISC). The RISC is capable of specifically binding to the mRNA of the target gene in cytoplasm, leading to the degradation of the mRNA of the target gene, so that the infected plant shows the mutation trait of the target gene, thus directly inferring the function of the target gene or providing indirect biological evidence. Under ongoing development and in depth research, the VIGS technology has been widely used in the research and identification of related functional genes such as plant resistance, growth and development, and metabolic regulation, and has a good development and application prospect in plant trait improvement and plant protection.

Fusarium graminearum gemytripvirus 1 (FgGMTV 1) is a multi-component single-stranded circular DNA virus, which contains three single-stranded circular DNA fragments: DNA-A, DNA-B and DNA-C, with molecular sizes of 1316 nt, 1320 nt and 1309 nt respectively and encoded with replication initiation protein (REP), Coat protein, CP) and unknown functional protein respectively. An infectious clone of FgGMTV1 has been successfully constructed in Mycovirus lab in Institute of Plant Protection (IPP), Chinese Academy of Agricultural Sciences (CAAS), and the relationship among the components is made clear: DNA-A and DNA-B are necessary for virus replication and infection, and their co-infection is capable of significantly inhibiting the growth, conidial production and pathogenicity of host fungi, but the virus is unstable and does not spread vertically through conidia; the replication and proliferation of DNA-C depend on DNA-A and DNA-B. The virus containing three components is stable and is capable of spreading through conidia, but it does not affect the phenotype and pathogenicity of host fungi. Therefore, it is possible to construct the vector of VIGS by artificial transfection with the infectious clone of FgGMTV1 as the basic element.

Wheat Fusarium head blight (FHB) is a worldwide wheat disease mainly caused by Fusarium graminearum complex. How to prevent the FHB and its toxin pollution is particularly urgent and important, which is directly related to food safety, food security and people's health. Chemical control is still an important measure for FHB control at present, but excessive dependence on chemical control leads to the resistance of pathogenic fungi to fungicides. In addition, due to the lack of wheat varieties with high resistance to the FHB, wheat FHB remains happening frequently at present and even for a long time to come. It is particularly important to find and explore new control methods of wheat FHB. By successfully constructing the VIGS vector of FgGMTV1 and silencing the genes related to host pathogenicity and toxin production, the obtained hypovirulent strains may be used as biological control factors, providing a new method for controlling the wheat FHB. Meanwhile, the obtained VIGS vector is capable of being widely used to study the gene function of Fusarium graminearum, which solves the problem that a gene knockout method fails to study the lethal genes.

### SUMMARY

The objective of the present disclosure is to provide a mycovirus-induced gene silencing vector and its construction method and disclosure, so as to solve the problems existing in the prior art. The gene silencing vector induced by Fusarium graminearum single-stranded circular DNA virus FgGMTV1 is capable of effectively reducing the toxin production and pathogenicity of infected Fusarium graminearum, and provide a new direction for preventing and controlling wheat Fusarium head blight (FHB).

In order to achieve the above objectives, the present disclosure provides the following scheme.

The disclosure provides a mycovirus-induced gene silencing vector, and a nucleotide sequence of the mycovirus-induced gene silencing vector is shown in SEQ ID NO: 2.

The disclosure also provides a construction method for the mycovirus-induced gene silencing vector, including following steps:

S1, connecting three single-stranded circular DNA molecules DNA-A, DNA-B and DNA-C of mycovirus FgGMTV1/HB58 in series and introducing into a same vector to construct a recombinant vector; and

S2, carrying out a deletion mutation on a coding protein p26 of the DNA-C molecule in the recombinant vector to obtain a mycovirus-induced gene silencing vector.

Optionally, 1.3 copies of the DNA-A, 1.3 copies of the DNA-B and 1.5 copies of the DNA-C are connected in series and then connected to pBluescript II SK(+) to construct the recombinant vector.

Optionally, a nucleotide sequence of the DNA-A is shown in SEQ ID NO: 5, a nucleotide sequence of the DNA-B is shown in SEQ ID NO: 6, and a nucleotide sequence of the DNA-C is shown in SEQ ID NO: 7.

Optionally, the deletion mutation is a deletion of a sequence of 454-603 nt of the coding protein p26 of the DNA-C.

The disclosure also provides a recombinant Fusarium graminearum including the mycovirus-induced gene silencing vector.

Optionally, the mycovirus-induced gene silencing vector carries exogenous genes. The exogenous genes include Tri101 gene and FgPP1 gene.

The disclosure also provides an application of the mycovirus-induced gene silencing vector or the recombinant Fusarium graminearum in preventing and controlling wheat FHB.

The disclosure also provides an application of the mycovirus-induced gene silencing vector or the recombinant Fusarium graminearum in preparing a preparation for preventing and controlling wheat FHB.

The disclosure discloses the following technical effects.

On the basis of previous research, the disclosure shortens the repetitive sequences of components DNA-A, DNA-B and DNA-C, and connects the three components in series to the same vector, and then selects the candidate mutant p26-D4 suitable as a VIGS vector by performing serial deletion mutation on the coding protein p26 of the DNA-C component. Through experimental verification, the constructed VIGS silencing vector p26-D4 is capable of accommodating 75-150 bp of exogenous genes, and effectively silence target genes such as GPF, Tri101 and FgPP1 in the wild Fusarium graminearum strain PH-1, which further verifies that this silencing vector p26-D4 carries exogenous genes to infect the Fusarium graminearum strain and is capable of effectively controlling wheat FHB. Therefore, the silencing vector p26-D4 provided by the disclosure offers a new direction for the prevention and control of wheat FHB.

### BRIEF DESCRIPTION OF THE DRAWINGS

FIG. 1 is a schematic structural diagram of an infectious clone pSK-ABC.

FIG. 2A shows Southern blot detection (results) of a pSK-ABC transfectant.

FIG. 2B shows a comparison of colony morphology of the pSK-ABC transfectant, a strain PH-1 and a strain A+B+C.

FIG. 2C shows a comparison of growth diameters of pSK-ABC transfectant, the strain PH-1 and the strain A+B+C on Potato Dextrose Agar (PDA) medium.

FIG. 2D shows a disease symptom of wheat spikelets when the pSK-ABC transfectant, the strain PH-1 and the strain A+B+C are inoculated onto wheat.

FIG. 2E shows the number of diseased spikelets per wheat head when the pSK-ABC transfectant, the strain PH-1 and the strain A+B+C are inoculated onto wheat.

FIG. 3A is a comparison of colony morphology of deletion mutants p26-D1, p26-D2, p26-D3, p26-D4 and p26-D5, the strain pSK-ABC and a strain PH-1(VF).

FIG. 3B shows Southern blot detection of the deletion mutants p26-D1, p26-D2, p26-D3, p26-D4 and p26-D5, the strain pSK-ABC and the strain PH-1(VF).

FIG. 3C shows a comparison of growth diameters of the deletion mutants p26-D1, p26-D2, p26-D3, p26-D4 and p26-D5, the strain pSK-ABC and the strain PH-1(VF) on PDA medium.

FIG. 4 is a schematic structural diagram of virus-induced gene silencing (VIGS) vector p26-D4.

FIG. 5A is a comparison of colony morphology, fluorescence observation and intensity analysis of a strain PH-1/WT(VF), a strain PH-1/GFP(VF) and strains infected by p26-D4, p26-D4-GFP75F, p26-D4-GFP75R, p26-D4-GFP150F and p26-D4-GFP150R.

FIG. 5B shows Southern blot detection of the stain PH-1/GFP(VF) and the strains infected by p26-D4, p26-D4-GFP75F, p26-D4-GFP75R, p26-D4-GFP150F, p26-D4-GFP150R, p26-D4-GFP300F, p26-D4-GFP300R, p26-D4-GFP450F and p26-D4-GFP450R.

FIG. 5C shows a comparison of GFP gene expression levels of the strains PH-1/WT (VF), PH-1/GFP(VF) and the strains infected by p26-D4, p26-D4-GFP75F, p26-D4-GFP75R, p26-D4-GFP150F and p26-D4-GFP150R.

FIG. 6A shows Southern blot detection of the strain PH-1/WT(VF), and the strains infected by p26-D4, p26-D4-Tri101 and p26-D4-FgPP1.

FIG. 6B shows a comparison of relative expression levels of Tri101 and FgPP1 genes in strains infected by p26-D4, p26-D4-Tri101 and p26-D4-FgPP1.

FIG. 6C shows a comparison of deoxynivaleno (DON) production induced by TBI-induced toxin-producing liquid medium among the strains infected by p26-D4, p26-D4-Tri101 and p26-D4-FgPP1.

FIG. 6D shows a comparison of colony morphology of the strain PH-1/WT(VF), and the strains infected by p26-D4, p26-D4-Tri101 and p26-D4-FgPP1.

FIG. 6E shows disease symptom of wheat spikelets after strains infected by p26-D4, p26-D4-Tri101 and p26-D4-FgPP1 are inoculated onto wheat.

FIG. 6F shows the number of diseased spikelets per wheat head after strains infected by p26-D4, p26-D4-Tri101 and p26-D4-FgPP1 are inoculated onto wheat.

FIG. 7A shows wheat FHB symptom after control of hypovirulent strains infected by p26-D4-Tri101 and p26-D4-FgPP1 measured by co-infection method (Test 1).

FIG. 7B shows the number of diseased spikelets per wheat head after control of hypovirulent strains infected by p26-D4-Tri101 and p26-D4-FgPP1 measured by co-infection method (Test 1).

FIG. 7C shows DON concentration in infected spikelets after control of hypovirulent strains infected by p26-D4-Tri101 and p26-D4-FgPP1 measured by co-infection method (Test 1).

FIG. 7D shows wheat FHB symptom after control of hypovirulent strains infected by p26-D4-Tri101 and p26-D4-FgPP1 measured by pre-spray method (Test 2).

FIG. 7E shows the number of diseased spikelets per wheat head after control of hypovirulent strains infected by p26-D4-Tri101 and p26-D4-FgPP1 measured by pre-spray method (Test 2).

FIG. 7F shows DON concentration in infected spikelets after control of hypovirulent strains infected by p26-D4-Tri101 and p26-D4-FgPP1 measured by pre-spray method (Test 2).

### DETAILED DESCRIPTION OF THE EMBODIMENTS

A number of exemplary embodiments of the present disclosure are now described in detail, and this detailed description should not be considered as a limitation of the present disclosure, but should be understood as a more detailed description of certain aspects, characteristics and embodiments of the present disclosure.

The Fusarium graminearum single-stranded DNA virus FgGMTV1 used in the embodiments of the present disclosure is isolated and identified by Mycovirus lab in Institute of Plant Protection (IPP), Chinese Academy of Agricultural Sciences (CAAS), see Li P. (2020). A tripartite ssDNA mycovirus from a plant pathogenic fungus is infectious as cloned DNA and purified viruses. Science Advances 6, eaay9634. The wild Fusarium graminearum strain PH-1 and the Fusarium graminearum strain PH-1/GFP with green fluorescent label are preserved in the Mycovirus lab in IPP, CAAS.

### Embodiment 1 Construction of an Infectious Clone pSK-ABC with Three Components of Fusarium graminearum DNA Virus FgGMTV1 in Series and Verification of its Infectivity

1. Construction of the Infectious Clone pSK-ABC with Three Components of DNA Virus FgGMTV1 in Series

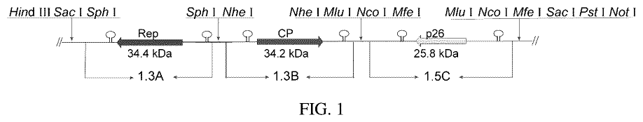

Patent CN109810997 A with a name of “Construction method of infectious clone of Fusarium graminearum single-stranded circular DNA virus FgGMTV1/HB58” discloses a construction method of infectious clone of Fusarium graminearum single-stranded DNA virus FgGMTV1/HB58, which includes DNA-A infectious clone construction, DNA-B infectious clone construction and DNA-C infectious clone construction. 2 units of DNA-A and DNA-B molecular sequences and 1.6 units of DNA-C molecular sequences are connected to the cloning vector pBluescript II SK(+) (pSK) respectively, and infectious clones pSK-2A, pSK-2B and pSK-1.6C of the three components of the virus are constructed. Because plasmids of three infectious clones transfect at the same time, and the steps are complicated, the DNA-A and DNA-B molecules herein are reduced to 1.3 units, and DNA-C molecules are reduced to 1.5 units, which only contain coding regions of the three components and two repetitive non-coding regions, respectively, and are connected in series to the vector pBluescript II SK(+). At the same time, suitable restriction enzyme sites are added at both ends of sequences of DNA-A, DNA-B and DNA-C respectively, so as to facilitate further mutant construction (see FIG. 1). Hind III (1-6 nt), SacI (7-12 nt), and SphI (13-18 nt) restriction enzyme sites are designated at one end of DNA-A. MluI (5464-5469 nt), NcoI (5470-5475 nt), MfeI (5476-5481 nt), SacI (5482-5487 nt), PstI (5488-5493 nt) and NotI (5494-5501 nt) restriction enzyme sites are designed at one end of DNA-C. The combination of Hind III (1-6 nt) and NotI (5494-5501 nt) is used to connect the clone to pBluescript II SK(+) cloning vector. Two restriction enzyme sites, SphI (1760-1765 nt) and NheI (1766-1771 nt), are designed between DNA-A and DNA-B, and four restriction enzyme sites, NheI (3509-3514 nt), MluI (3515-3520 nt), NcoI (3521-3526 nt) and MfeI (3527-3532 nt), are designed between DNA-B and DNA-C. Through the rational use of various restriction enzyme sites, the mutation of the three components of DNA-A, DNA-B and DNA-C is realized. The infectious clone sequence is synthesized by Shanghai Generay Biotechnology Co., Ltd. and connected to pBluescript II SK(+) cloning vector to obtain the infectious clone pSK-ABC. The sequence shown in the following SEQ ID NO: 1 is the complete sequence of the infectious clone pSK-ABC (without vector), with restriction enzyme sites of 1-18 nt, 1760-1771 nt, 3509-3532 nt, 5464-5501 nt. 19-1759 nt is the sequence of component A, 1772-3508 nt is the sequence of component B and 3533-5463 nt the sequence of component C. Hind III (1-6 nt) and NotI (5494-5501 nt) double restriction enzyme digestion are used to connect the sequence to pBluescript II SK(+) cloning vector. SEQ ID NO: 1 is:

5′-AAGCTTGAGCTCGCATGCTTCGGCGAGTTTAGTTGCGTCGAACTCATCAGGTGTTTGGGA
ATAAGTGAAAAGCCATTTTTTACATCGGAGTCGGTATCTGTGATCACTAGTTAGTTA
GTTGGGAATAGCTTCACTTTTTGGCAATTGAGTTAAGTAAGGGTCATTGCACCATCAT
GGGGGAGGGACCTCCGGGGGGGCCCCATGTGGGGCTGTACCCGCGTCAGAAGCCCA
AGCTCTGGAATAAAAAAAATAAAAAATACGTACTGGTTGCGTTGTCGTTTTTGTGGCT
GAGGTGTTTCTGAGTCACATGATTCGGAAGCGCGTTTGGACATTGTGGCTGAAGAGT
GGCTTTGCGGAGGTCAAGAAATCTAAGAAAGAGAGGATTTAAGATTCTTCTCTTTCTT
AGATTTGTGACCGTGACCCAAGTTTGATTTATATACTAGTTATGTGTTGACTAAGGTAC
AGAGAGGGGTAGTGATCTCCACAATAATGGCGTTAGCGTCCATCCATTTCCAATCAAT
ATCAGTGCAGGTACGGCGTGGATCTTCATTGGCTATGTAGATGCACGGCTTACCCCAC
ATGACCCGCTGTTTTCCTTTGTACTTGTCGGTAGCTACGAATTCTCGTTGGCCGAACC
ATTGTTTGTAATTTGGAAAAGAGGAGAACCCACCAATCAAGTCATCAAACACCGCGT
AGTTTACGTTAGGGTGAAAGTCAGAGAGCATGAACATGCCTGGGAAGTAGGCGTGAT
TGGCTAGACTTCGCGCCCATAGAGTTTTACCGAGGCGGGAAGCGCCGAATAAAATGA
GACTTATGGGTCGGTATGATGTGTTGTTCGTGACGTAATTGTCAACCCAGTCTTTGAG
TTCGGGATAGTCAGAGTAATTGACAGTAATTTGTGGAGATTCGTATGTGCTCTGTGGT
GGACCGAATTTCCATTCAGCAAATGACTTGATGTTGTTCCAGCATTTGATGGATTGGTT
AGGTAGTTCGGTGGCAGCCAAGTGTAAAAATTCTTTTGCGGAAGTTGATTGGTCGAT
GATACGCAACCATTCTTTATCCATGTTCTTTTGGTTCAAGGATGGTTGGTGTGGGGGTG
TACCCTCTTCCCATTGGATGTCGTTATCTTTTTTTACGTAATTGACGACAGTGTGTGGG
GTTCTGGTGACAACTTCGATGTTGGGGTGAACACCACAGAAATCGAAGTCCCGTGCG
TTGTTTGAAGTGTGAATAGTTTCAACTTCCCAATACACGTGATGGTGGAATCCGCCAT
CTTTGTGTTGTTCTTTTGAGATGACGAGATAGACAAGAAGTGGACTTTTTTCTTTGAA
CATTTCGGCGAGTTTAGTTGCGTCGAACTCATCAGGTGTTTGGGAATAAGTGAAAAG
CCATTTTTTACATCGGAGTCGGTATCTGTGATCACTAGTTAGTTAGTTGGGAATAGCTT
CACTTTTTGGCAATTGAGTTAAGTAAGGGTCATTGCACCATCATGGGGGAGGGACCTC
CGGGGGGGCCCCATGTGGGGCTGTACCCGCGTCAGAAGCCCAAGCTCTGGAATAAA
AAAAATAAAAAATACGTACTGGTTGCGTTGTCGTTTTTGTGGCTGAGGTGTTTCTGAG
TCACATGATTCGGAAGCGCGTTTGGACATTGTGGCTGAAGAGTGGCTTTGCGGAGGT
CAAGAAATCTAAGAAAGAGAGGATTTAAGATTCTTCTCTTTCTTAGATTTGTGACCGT
GACCCAAGTTTGATTTATATACTAGGCATGC
GCTAGCGGTAATTTTAGTGTAGCAAAATTGAGTTGGTGATAGCTTCATTTTTTTGGATC
CACTTTGTGACAATTGAGTTGAAGTAAGGGTCATTGCACCATCATGGGGGAGGGACC
TCCGGGGGGGCCCCATGTGGGGCTGTACCCGCGTCAGAAGCCCAAGCTCCGGAATAA
AAAAAAATCAAAAGTAGACTGTCGGTTAAAGTTCGGTTGGGTAGGATTAGTCAGCAA
ATTTTCAACCAATAGCGGAGGTCAAGAAATCTAAGAAAGAGAGGATTTAAGATTCTT
CTCTTTCTTAGATTTGTGACCGTGACCCAGGTTTGATTTTGGGTATATAAGGGAGGGGT
AGCCACCATTTTTGCTAGTCTGTTTTTGGACTTAAAAAATTTATTTTTTAACACAAAAC
ATTATTACGTCGACAAAAATGGCTTCTACAAAGAAGAAATCATACAACAACAAGAAG
GCTTATAAAAAAAAAGAATGGAAGTCGAAGAAGACTTGGGACAAGTCTAGTTATTAC
GACAATTACCAGTCGAAGATGAATATTTCGAATATGCAGACGAAGAGGGACAACATG
ATGTGTGTGACGTCACATTGTGGTGTTCCGAATGCGGCGTTACTGGAGAATTCTGTTG
TGGGTGAAATTCCAGCCAATATGGGAGTTCATTATATTATGTGGTCTCCTACGTATCGA
GAGGCGGTACCACCGAATCGAGCGGCACAGTTGGATCGGCAATCCGCAAACACATTT
TTTACTGGTTGGAAGGATAATTTGTCCTATCAATTTAAGGGACAGATTACAGGGATTCA
CCTGAGGGTTGTGATATCTACCCGAAGAGAAGTGGAGTCCGCGCAGCCTTTTATTGG
GCCGGGGAATACGCTGTGCAGAAACTTGGCGGTTCGTGATATGTCGGATGAGACATT
GGACCAGTTTTTGTCGGGTACCCGGGATGTTGATTGGACGTTGGTGAATGTGATGGAC
ACGATGTTTGATCCGGCGGTGTGCAAGGTGTTGTTTCGGCAGAGGAAGATTTTAGGT
GCAGCTGATGCGTTGTTGAAGACGGAGGAGTTTTATCACCGTATCCGTCGGCCTATGG
TGTACGGCGATAGGCAGGATGGTTTGGAGTTTGTGTCTAGTGGTTGGGCTGGAAGGG
AGTCGGAGAACATATACGTCATTGATATGTACTCTTTGATTTCGGCAGCCCCACCGTTA
GGTAATTTGTTGGATGGAGAGGGAAATATTGTTTTGGATGACAAGAAACGGCCTATTC
CCGTATATGCGAAGTTAAATATTAGTGGAAATAGTATAGTGTATTGGAGGGAGTAGGGT
AATTTTAGTGTAGCAAAATTGAGTTGGTGATAGCTTCATTTTTTTGGATCCACTTTGTG
ACAATTGAGTTGAAGTAAGGGTCATTGCACCATCATGGGGGAGGGACCTCCGGGGGG
GCCCCATGTGGGGCTGTACCCGCGTCAGAAGCCCAAGCTCCGGAATAAAAAAAAATC
AAAAGTAGACTGTCGGTTAAAGTTCGGTTGGGTAGGATTAGTCAGCAAATTTTCAAC
CAATAGCGGAGGTCAAGAAATCTAAGAAAGAGAGGATTTAAGATTCTTCTCTTTCTTA
GATTTGTGACCGTGACCCAGGTTTGATTTTGGGTATATAAGGGAGGGGTAGCCACCAT
TTTTGCTAGTCTGTTTTTGGACTTAAAAAATTTATTTTTTAACACAAAACATTATTACGT
CGACAAAAGCTAGC ACGCGT CCATGG
CAATTGCTTTATATTGTAAAAAATATTTGTAACTGTAAATAATTAGTTGGTGATAGCTTC
ATTTTTTTTACTCCACTTTGTGACAATTGAGTTAAGTAAGGGTCATTGCACCATCATGG
GGGAGGGACCTCCGGGGGGGCCCCATGTGGGGCTGTACCCGCGTCAGAAGCCCAAG
CTCTTTGTTGAGCCGAGCGCAGCGGTAATTTGGAGTCACGTGAGGTAAAATAAAATG
TGGACTTACGTTCTTGGAATTGATGATTGAGACATTTTGAAAAAGTGTTGGAGTGGTT
GGGGTATTTATGGTCAAGGACATGTTTGGTGGTGTCATTGGTTAATATAGGTACTGTCG
GTAGATAGTTGTTGCGGTTGAAGTATAATGCGTGGAGCACCGAGGTCAGAACTTTTAG
GAAAGACGATTTAAGATTCTCTTTCCTAAAAGTCTGACCGTGACTCCCTTTGGCCTTG
ACGACGTTATTGGTGGAGGATTGGAATGTTACCCGCAATTTCACGTGACATGTGGAAA
TGTGGTGACATGAAGAATTGTGGGACGGCACAATTTTAATTGGGTGGAACACAGCAG
GGTAGGATTAGGCAGAATGAGGCAGATTTAGGCAGCGGAAATTTATTTTTAAATTGGA
GCATTGTCTAAATCTAGAAGTACATGGTACCAGTCATCATCGTTTGGTTGATGATCTGG
TATGTCCACCCAATCAGGATCATTTAAATTATGTACAATTGAATTGTTATGATTTGTAAA
AAAAAAAGATAGGTAATCGCATAACGTGTTTTTGTTGAATAAAATAATACACGTGTTT
GCGTATTGTTGTACCATGTCATGTGGGTCGGGATTGTTGTGGAGATGACACGTGTTAG
TGCTGGTATGAACTCGCAGGAGTGTAGTACCAGTCGACAAAGGCTTGGATGTGTTGG
CGGTGTCGTTGGAGTAGTGTAGGGTGAGTTTCCTCGAAACGACTAAGAAACGCCCCT
TGATTTGGCTCGGAGAAAAGTTGTTGCCAGAAGGGGTCAGATGTGGCTGAGCAAAG
ATCTGCATCCCCGAACTGAGCCACAAGCACCTCTTTAGACTGGATGCTCTCGTATACC
GGGCCTGCATGTGTGTATATTGGCAGAATAGAAGGCATAACTCCGCTGTAAGCGAGTT
GTAGGCTTCGTTGGAGGGGAATGTTTCCAGTATCGATGTAGATATCGAGATCACAGTT
CCCATTAGTTCTTGTATTGTAACCGGCGATGAATCTCTTTGGATTGATGTCCATTTGGA
GTTGAGAAGTGAAATTATCTGATTGTTTTGGGTCGACATCTTTATATTGTAAAAAATAT
TTGTAACTGTAAATAATTAGTTGGTGATAGCTTCATTTTTTTTACTCCACTTTGTGACAA
TTGAGTTAAGTAAGGGTCATTGCACCATCATGGGGGAGGGACCTCCGGGGGGGCCCC
ATGTGGGGCTGTACCCGCGTCAGAAGCCCAAGCTCTTTGTTGAGCCGAGCGCAGCGG
TAATTTGGAGTCACGTGAGGTAAAATAAAATGTGGACTTACGTTCTTGGAATTGATGA
TTGAGACATTTTGAAAAAGTGTTGGAGTGGTTGGGGTATTTATGGTCAAGGACATGTT
TGGTGGTGTCATTGGTTAATATAGGTACTGTCGGTAGATAGTTGTTGCGGTTGAAGTAT
AATGCGTGGAGCACCGAGGTCAGAACTTTTAGGAAAGACGATTTAAGATTCTCTTTC
CTAAAAGTCTGACCGTGACTCCCTTTGGCCTTGACGACGTTATTGGTGGAGGATTGGA
ATGTTACCCGCAATTTCACGTGACATGTGGAAATGTGGTGACATGAAGAATTGTGGGA
CGGCACAATTTTAATTGGGTGGAACACAGCAGGGTAGGATTAGGCAGAATGAGGCAG
ATTTAGGCAGCGGAAATTTATTTACGCGT CCATGG CAATTG GAGCTC CTGCAG
GCGGCCGC-3′.

2. Protoplast Preparation of Fusarium graminearum

Fusarium graminearum strain PH-1 on PDA plate is inoculated into a 250 mL triangular flask with 100 mL carboxylmethyl cellulose (CMC) liquid medium, and cultured for 3-5 days at 25° C. and 180 rpm (preferably 4 days); the culture medium is filtered with sterilized three-layer lens-wiping paper, centrifuged at 4000 rpm for 5 min at room temperature, and the supernatant is discarded; sterilized water is added, conidia precipitation is resuspended and diluted to the concentration of 10⁷ spores/mL; 3×10⁷ spores are transferred to a 250 mL triangular flask containing 100 mL Yeast Extract Peptone Dextrose (YEPD) liquid medium, and shaken at 25° C. and 180 rpm for 12-14 h; the overnight culture medium is filtered with three-layer sterilized lens-wiping paper, rinsed with sterile water for three times, then rinsed with 1 M sucrose for two times, drained with sterilized filter paper, and mycelium is collected; mycelium is transferred to 20 mL of protoplast hydrolysate with a sterile tweezers, gently shaken at 90 rpm and 28° C. for 2-3 h, and the protoplast granules are examined in terms of quantity by microscope every 0.5 h; the enzymolysis mixture is filtered with two-layer sterilized lens-wiping paper into a sterile 50 mL centrifuge tube, and rinsed twice with 1 M sucrose solution to rinse off as many protoplasts as possible; the protoplasts are centrifuged at 2600 rpm at 4° C. for 1 min, the supernatant is discarded, the protoplast are resuspended with Sucrose-Tris-Calcium (STC) buffer (1 M sorbitol, 50 mM Tris-HCl pH 8.0, and 50 mM CaCl₂·2H₂O) precooled on ice, and the protoplast are washed for three times according to the above centrifugation conditions; finally, the protoplasts are resuspended with STC buffer, and the final concentration of the protoplasts is adjusted to 2×10⁷ cells/mL, the protoplasts are sub-packaged at 200 μL/tube, added with dimethyl sulfoxide (DMSO) with final concentration of 7%, mixed well and stored at −80° C.

3. Polyethylene Glycol (PEG)-Mediated Transfection of pSK-ABC

The protoplasts are taken out at −80° C. and thawed on ice; 200 μL PH-1 protoplasts and 30 μg plasmid are added into a 2 mL centrifuge tube, mixed gently, and placed on ice for 20 min; 1.25 mL PEG-Tris-Calcium (PTC) buffer (STC buffer containing 40% PEG 4000) is dripped into the 2 mL centrifuge tube, mixed with a gun head, and then allowed to stand at room temperature for 20 min. The protoplasts in the 2 mL centrifuge tube are transferred into the 15 mL centrifuge tube, the 2 mL centrifuge tube is rinsed repeatedly with 5 mL Terrific Broth 3 (TB3) (3 g Yeast Extract, 3 g Casamino Acids, 200 g Sucrose, added with distilled water to constant volume to 1 L) (containing 50 μg/mL ampicillin), then added into the above 15 mL centrifuge tube, shaken lightly at 25° C. and 90 rpm overnight (16 h); the regenerated substance of protoplasts are centrifuged at 4000 rpm for 5 min, the supernatant is discarded, 600 μL STC is added to resuspend and precipitate, 200 μL of the protoplasts are taken respectively in turn and put in the center of a 9 cm culture dish, and added with 20 mL of TB3 solid medium containing 50 ug/mL ampicillin; the plate is placed with face upward, and cultured overnight at 25° C. in the dark. The next day, the plate is tilted to remove STC floating on the surface, and cultured for 5 days. The morphology of mycelium is observed, small pieces of myceliums is cut from different positions on the edge and transferred to PDA plate, cultured, and further experimental observation is conducted after molecular identification.

### 4. Determination of Colony Morphology and Growth Rate of the Strains

The mycelian blocks of transfectant strain are punched with a 5 mm punch and placed in the center of PDA medium plate to activate the strain. A 5 mm punch is used to punch the blocks on the edge of the colony of the activated strain, and the blocks are transferred to the center of the PDA medium plate, and 5 repetitions are conducted respectively. After 4 days of dark culture at 25° C., the colony morphology and diameter are observed and statistically analyzed.

### 5. Determination of Pathogenicity of the Strains

Yangmai 158, a wheat variety, is planted and used for wheat spikelet inoculation when the wheat grows to the flowering stage. The conidia of each transfectant strain are prepared according to the above method, and the spore concentration is measured by blood cell counting plate, and then the spore concentration is adjusted to 3×10⁵ spores/mL. Spikelets with basically the same growth are selected, and when the fifth spikelet is found from bottom to top, the inner glume from the outer glume are separated with fingers. 10 μL of the prepared spore solution is sucked and injected into the root between the inner glume and the outer glume, and spikelet is gently closed to keep a natural state. The inoculated spikelet is marked with a marker and a label is put on the wheat stalk, and the inoculation strain, inoculation method, inoculation date, inoculator and other information are recorded. Each strain is inoculated onto at least 15 spikelets, subjected to moisturizing culture with fresh-keeping bag for 48 h; after taking off the fresh-keeping bag, each strain is cultured for 14 days, the symptom of wheat spikelets is observed, and the number of diseased spikelets per wheat head is counted.

### 6. Results

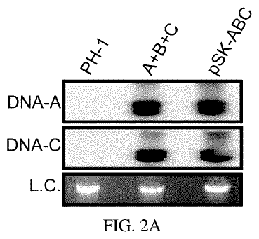

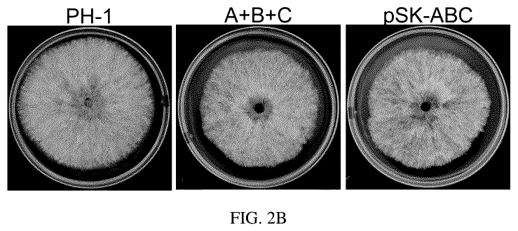

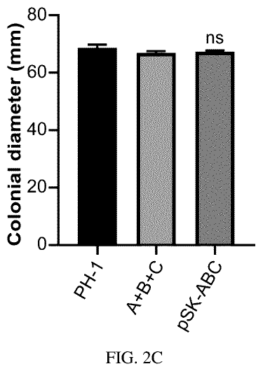

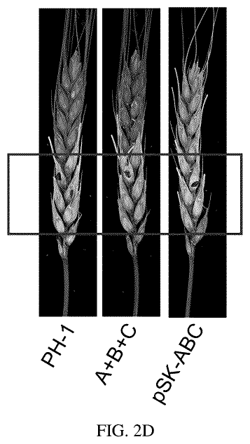

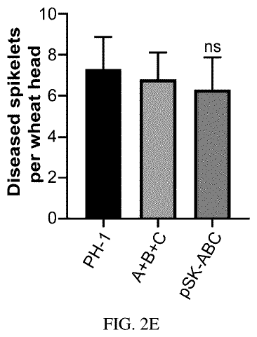

FIG. 2A, FIG. 2B, FIG. 2C, FIG. 2D, and FIG. 2E show an infection activity analysis of an infectious clone pSK-ABC of FgGMTV1 with three components connected in series. The results show that the pSK-ABC transfectant strain is obtained through protoplast preparation, transfection and regeneration. By Southern blot analysis (see FIG. 2A), it is confirmed that the virus exists in the infectious clone pSK-ABC transfectant strain, and there is no significant difference in colony morphology, colony growth rate and pathogenicity between the pSK-ABC transfectant strain and the strain A+B+C co-transfected by infectious clones of three components (see FIG. 2B-FIG. 2E). It is confirmed that the infectious clone pSK-ABC with three components of DNA virus FgGMTV1 in series is successfully constructed.

### Embodiment 2 Construction of Gene Silencing Vector p26-D4 Based on Virus FgGMTV1

### 1. Design and Construction of Gene Silencing Vector Based on FgGMTV1

Based on the infectious clone pSK-ABC, the p26 coding frame of DNA-C component is deleted with the length of 150 nt in turn, and five deletion mutants are obtained, named p26-D1, p26-D2, p26-D3 and p26-D4, and p26-D5. The p26-D5 is deleted by 81 nt. The method of constructing these mutants is completed by KOD-Plus-Mutagenesis Kit (code NO. SMK-101) of Toyobo Life Science company. The experimental principle of the kit is that by using pSK-ABC as a template through inverse PCR method, two primers are designed in the opposite direction, and restriction enzyme sites NsiI and AgeI are introduced at the same time, and the plasmid is subjected to complete PCR by using high-fidelity KOD-Plus-enzyme, and finally the target mutants are obtained by screening.

The deletion mutants based on DNA-C are constructed by designing specific primers, as shown in Table 1, in which the underlined sequences are introduced restriction enzyme sites NsiI and AgeI.

TABLE 1
DNA-C mutant primers

| Primer name | Primer sequence (5′-3′) |
|---|---|
| p26-D1-F | AGGATGCATTACAACTCGCTTACAGCGGAGTTA |
| (SEQ ID NO: 8) | TGCCTT |
| p26-D1-R | AGGACCGGTCATCTTTATATTGTAAAAAATATT |
| (SEQ ID NO: 9) | TGTAAC |
| p26-D2-F | AGGATGCATAACAACTTTTCTCCGAGCCAAATC |
| (SEQ ID NO: 10) | AAGGGG |
| p26-D2-R | AGGACCGGTGGCTTCGTTGGAGGGGAATGTTTC |
| (SEQ ID NO: 11) | CAGTAT |
| p26-D3-F | AGGATGCATCATCTCCACAACAATCCCGACCCA |
| (SEQ ID NO: 12) | CATGAC |
| p26-D3-R | AGGACCGGTGCCAGAAGGGGTCAGATGTGGCTG |
| (SEQ ID NO: 13) | AGCAAA |
| p26-D4-F | AGGATGCATGATTGGGTGGACATACCAGATCAT |
| (SEQ ID NO: 14) | CAACCA |
| p26-D4-R | AGGACCGGTACACGTGTTAGTGCTGGTATGAAC |
| (SEQ ID NO: 15) | TCGCAG |
| p26-D5-F | AGGATGCATTAAAAATAAATTTCCGCTGCCTAA |
| (SEQ ID NO: 16) | ATCTGC |
| p26-D5-R | AGGACCGGTAGGATCATTTAAATTATGTACAAT |
| (SEQ ID NO: 17) | TGAATT |

Protoplast preparation of Fusarium graminearum, transfection of virus deletion mutants mediated by PEG and observation of colony morphology of the strains are all the same as those in Embodiment 1.

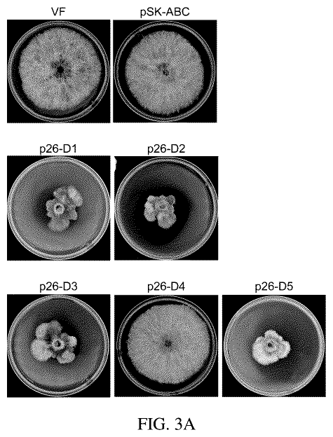

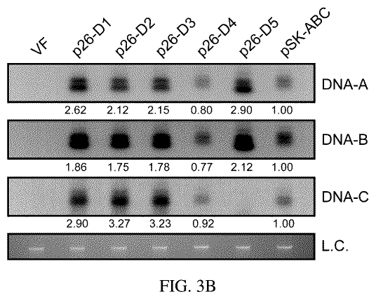

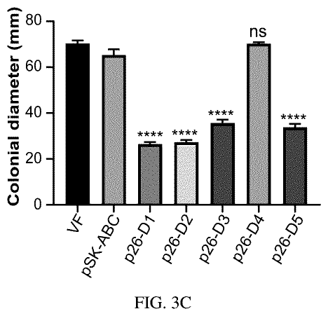

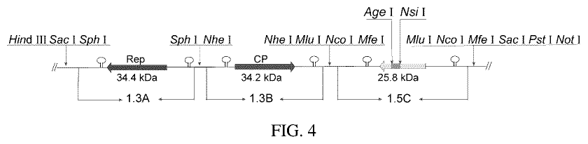

FIG. 3A, FIG. 3B, and FIG. 3C illustrate an analysis of infection activity of deletion mutant based on DNA-C. The results show that five deletion mutants p26-D1, p26-D2, p26-D3, p26-D4 and p26-D5 are constructed based on the infectious clone pSK-ABC. It is confirmed by Southern blot that three components of the mutants p26-D1, p26-D2, p26-D3 and p26-D4 could be effectively replicated, but only p26-D4 has the same phenotype as the wild-type virus, that is, the infection of mutant virus would not cause abnormal phenotype of the host (FIG. 3A, FIG. 3B, FIG. 3C). p26-D4 may be used as a candidate gene silencing vector, named p26-D4, that is, the sequence of 284-434 nt of the coding protein p26 of the DNA-C component is deleted, and the restriction enzyme sites NsiI and AgeI are introduced, the structure of the VIGS vector p26-D4 is shown in FIG. 4.

The complete sequence (without vector) of the VIGS vector p26-D4 is shown in SEQ ID NO: 2, with the restriction enzyme sites of 1-18 nt, 1760-1771 nt, 3509-3532 nt, 4239-4256 nt, 5332-5369 nt. 19-1759 nt is the sequence of component A, 1772-3508 nt is the sequence of component B and 3533-4238 nt, 4257-5331 nt is the sequence of component C. Hind III (1-6 nt) and NotI (5362-5369 nt) are used to connect the sequence to pBluescript II SK(+) cloning vector. NsiI (4239-4244 nt) and AgeI (4251-4256 nt) are used to insert foreign gene fragments. SEQ ID NO: 2 is:

5′-AAGCTT GAGCTC GCATGCTTCGGCGAGTTTAGTTGCGTCGAACTCATCAGGTGTTTGGGAATAAGTGAA
AAGCCATTTTTTACATCGGAGTCGGTATCTGTGATCACTAGTTAGTTAGTTGGGAATAG
CTTCACTTTTTGGCAATTGAGTTAAGTAAGGGTCATTGCACCATCATGGGGGAGGGAC
CTCCGGGGGGGCCCCATGTGGGGCTGTACCCGCGTCAGAAGCCCAAGCTCTGGAATA
AAAAAAATAAAAAATACGTACTGGTTGCGTTGTCGTTTTTGTGGCTGAGGTGTTTCTG
AGTCACATGATTCGGAAGCGCGTTTGGACATTGTGGCTGAAGAGTGGCTTTGCGGAG
GTCAAGAAATCTAAGAAAGAGAGGATTTAAGATTCTTCTCTTTCTTAGATTTGTGACC
GTGACCCAAGTTTGATTTATATACTAGTTATGTGTTGACTAAGGTACAGAGAGGGGTA
GTGATCTCCACAATAATGGCGTTAGCGTCCATCCATTTCCAATCAATATCAGTGCAGGT
ACGGCGTGGATCTTCATTGGCTATGTAGATGCACGGCTTACCCCACATGACCCGCTGT
TTTCCTTTGTACTTGTCGGTAGCTACGAATTCTCGTTGGCCGAACCATTGTTTGTAATT
TGGAAAAGAGGAGAACCCACCAATCAAGTCATCAAACACCGCGTAGTTTACGTTAGG
GTGAAAGTCAGAGAGCATGAACATGCCTGGGAAGTAGGCGTGATTGGCTAGACTTCG
CGCCCATAGAGTTTTACCGAGGCGGGAAGCGCCGAATAAAATGAGACTTATGGGTCG
GTATGATGTGTTGTTCGTGACGTAATTGTCAACCCAGTCTTTGAGTTCGGGATAGTCA
GAGTAATTGACAGTAATTTGTGGAGATTCGTATGTGCTCTGTGGTGGACCGAATTTCC
ATTCAGCAAATGACTTGATGTTGTTCCAGCATTTGATGGATTGGTTAGGTAGTTCGGTG
GCAGCCAAGTGTAAAAATTCTTTTGCGGAAGTTGATTGGTCGATGATACGCAACCATT
CTTTATCCATGTTCTTTTGGTTCAAGGATGGTTGGTGTGGGGGTGTACCCTCTTCCCAT
TGGATGTCGTTATCTTTTTTTACGTAATTGACGACAGTGTGTGGGGTTCTGGTGACAA
CTTCGATGTTGGGGTGAACACCACAGAAATCGAAGTCCCGTGCGTTGTTTGAAGTGT
GAATAGTTTCAACTTCCCAATACACGTGATGGTGGAATCCGCCATCTTTGTGTTGTTCT
TTTGAGATGACGAGATAGACAAGAAGTGGACTTTTTTCTTTGAACATTTCGGCGAGTT
TAGTTGCGTCGAACTCATCAGGTGTTTGGGAATAAGTGAAAAGCCATTTTTTACATCG
GAGTCGGTATCTGTGATCACTAGTTAGTTAGTTGGGAATAGCTTCACTTTTTGGCAATT
GAGTTAAGTAAGGGTCATTGCACCATCATGGGGGAGGGACCTCCGGGGGGGCCCCAT
GTGGGGCTGTACCCGCGTCAGAAGCCCAAGCTCTGGAATAAAAAAAATAAAAAATAC
GTACTGGTTGCGTTGTCGTTTTTGTGGCTGAGGTGTTTCTGAGTCACATGATTCGGAA
GCGCGTTTGGACATTGTGGCTGAAGAGTGGCTTTGCGGAGGTCAAGAAATCTAAGAA
AGAGAGGATTTAAGATTCTTCTCTTTCTTAGATTTGTGACCGTGACCCAAGTTTGATTT
ATATACTAGGCATGCGCTAGCGGTAATTTTAGTGTAGCAAAATTGAGTTGGTGATAGCT
TCATTTTTTTGGATCCACTTTGTGACAATTGAGTTGAAGTAAGGGTCATTGCACCATCA
TGGGGGAGGGACCTCCGGGGGGGCCCCATGTGGGGCTGTACCCGCGTCAGAAGCCC
AAGCTCCGGAATAAAAAAAAATCAAAAGTAGACTGTCGGTTAAAGTTCGGTTGGGTA
GGATTAGTCAGCAAATTTTCAACCAATAGCGGAGGTCAAGAAATCTAAGAAAGAGAG
GATTTAAGATTCTTCTCTTTCTTAGATTTGTGACCGTGACCCAGGTTTGATTTTGGGTA
TATAAGGGAGGGGTAGCCACCATTTTTGCTAGTCTGTTTTTGGACTTAAAAAATTTATT
TTTTAACACAAAACATTATTACGTCGACAAAAATGGCTTCTACAAAGAAGAAATCATA
CAACAACAAGAAGGCTTATAAAAAAAAAGAATGGAAGTCGAAGAAGACTTGGGACA
AGTCTAGTTATTACGACAATTACCAGTCGAAGATGAATATTTCGAATATGCAGACGAA
GAGGGACAACATGATGTGTGTGACGTCACATTGTGGTGTTCCGAATGCGGCGTTACT
GGAGAATTCTGTTGTGGGTGAAATTCCAGCCAATATGGGAGTTCATTATATTATGTGGT
CTCCTACGTATCGAGAGGCGGTACCACCGAATCGAGCGGCACAGTTGGATCGGCAAT
CCGCAAACACATTTTTTACTGGTTGGAAGGATAATTTGTCCTATCAATTTAAGGGACA
GATTACAGGGATTCACCTGAGGGTTGTGATATCTACCCGAAGAGAAGTGGAGTCCGC
GCAGCCTTTTATTGGGCCGGGGAATACGCTGTGCAGAAACTTGGCGGTTCGTGATATG
TCGGATGAGACATTGGACCAGTTTTTGTCGGGTACCCGGGATGTTGATTGGACGTTGG
TGAATGTGATGGACACGATGTTTGATCCGGCGGTGTGCAAGGTGTTGTTTCGGCAGA
GGAAGATTTTAGGTGCAGCTGATGCGTTGTTGAAGACGGAGGAGTTTTATCACCGTAT
CCGTCGGCCTATGGTGTACGGCGATAGGCAGGATGGTTTGGAGTTTGTGTCTAGTGGT
TGGGCTGGAAGGGAGTCGGAGAACATATACGTCATTGATATGTACTCTTTGATTTCGG
CAGCCCCACCGTTAGGTAATTTGTTGGATGGAGAGGGAAATATTGTTTTGGATGACAA
GAAACGGCCTATTCCCGTATATGCGAAGTTAAATATTAGTGGAAATAGTATAGTGTATT
GGAGGGAGTAGGGTAATTTTAGTGTAGCAAAATTGAGTTGGTGATAGCTTCATTTTTT
TGGATCCACTTTGTGACAATTGAGTTGAAGTAAGGGTCATTGCACCATCATGGGGGAG
GGACCTCCGGGGGGGCCCCATGTGGGGCTGTACCCGCGTCAGAAGCCCAAGCTCCG
GAATAAAAAAAAATCAAAAGTAGACTGTCGGTTAAAGTTCGGTTGGGTAGGATTAGT
CAGCAAATTTTCAACCAATAGCGGAGGTCAAGAAATCTAAGAAAGAGAGGATTTAAG
ATTCTTCTCTTTCTTAGATTTGTGACCGTGACCCAGGTTTGATTTTGGGTATATAAGGG
AGGGGTAGCCACCATTTTTGCTAGTCTGTTTTTGGACTTAAAAAATTTATTTTTTAACA
CAAAACATTATTACGTCGACAAAAGCTAGCACGCGTCCATGGCAATTGCTTTATATTGT
AAAAAATATTTGTAACTGTAAATAATTAGTTGGTGATAGCTTCATTTTTTTTACTCCACT
TTGTGACAATTGAGTTAAGTAAGGGTCATTGCACCATCATGGGGGAGGGACCTCCGG
GGGGGCCCCATGTGGGGCTGTACCCGCGTCAGAAGCCCAAGCTCTTTGTTGAGCCGA
GCGCAGCGGTAATTTGGAGTCACGTGAGGTAAAATAAAATGTGGACTTACGTTCTTG
GAATTGATGATTGAGACATTTTGAAAAAGTGTTGGAGTGGTTGGGGTATTTATGGTCA
AGGACATGTTTGGTGGTGTCATTGGTTAATATAGGTACTGTCGGTAGATAGTTGTTGCG
GTTGAAGTATAATGCGTGGAGCACCGAGGTCAGAACTTTTAGGAAAGACGATTTAAG
ATTCTCTTTCCTAAAAGTCTGACCGTGACTCCCTTTGGCCTTGACGACGTTATTGGTG
GAGGATTGGAATGTTACCCGCAATTTCACGTGACATGTGGAAATGTGGTGACATGAA
GAATTGTGGGACGGCACAATTTTAATTGGGTGGAACACAGCAGGGTAGGATTAGGCA
GAATGAGGCAGATTTAGGCAGCGGAAATTTATTTTTAAATTGGAGCATTGTCTAAATCT
AGAAGTACATGGTACCAGTCATCATCGTTTGGTTGATGATCTGGTATGTCCACCCAATC
ATGCATCCTAGGACCGGTACACGTGTTAGTGCTGGTATGAACTCGCAGGAGTGTAGTA
CCAGTCGACAAAGGCTTGGATGTGTTGGCGGTGTCGTTGGAGTAGTGTAGGGTGAGT
TTCCTCGAAACGACTAAGAAACGCCCCTTGATTTGGCTCGGAGAAAAGTTGTTGCCA
GAAGGGGTCAGATGTGGCTGAGCAAAGATCTGCATCCCCGAACTGAGCCACAAGCA
CCTCTTTAGACTGGATGCTCTCGTATACCGGGCCTGCATGTGTGTATATTGGCAGAATA
GAAGGCATAACTCCGCTGTAAGCGAGTTGTAGGCTTCGTTGGAGGGGAATGTTTCCA
GTATCGATGTAGATATCGAGATCACAGTTCCCATTAGTTCTTGTATTGTAACCGGCGAT
GAATCTCTTTGGATTGATGTCCATTTGGAGTTGAGAAGTGAAATTATCTGATTGTTTTG
GGTCGACATCTTTATATTGTAAAAAATATTTGTAACTGTAAATAATTAGTTGGTGATAGC
TTCATTTTTTTTACTCCACTTTGTGACAATTGAGTTAAGTAAGGGTCATTGCACCATCA
TGGGGGAGGGACCTCCGGGGGGGCCCCATGTGGGGCTGTACCCGCGTCAGAAGCCC
AAGCTCTTTGTTGAGCCGAGCGCAGCGGTAATTTGGAGTCACGTGAGGTAAAATAAA
ATGTGGACTTACGTTCTTGGAATTGATGATTGAGACATTTTGAAAAAGTGTTGGAGTG
GTTGGGGTATTTATGGTCAAGGACATGTTTGGTGGTGTCATTGGTTAATATAGGTACTG
TCGGTAGATAGTTGTTGCGGTTGAAGTATAATGCGTGGAGCACCGAGGTCAGAACTTT
TAGGAAAGACGATTTAAGATTCTCTTTCCTAAAAGTCTGACCGTGACTCCCTTTGGCC
TTGACGACGTTATTGGTGGAGGATTGGAATGTTACCCGCAATTTCACGTGACATGTGG
AAATGTGGTGACATGAAGAATTGTGGGACGGCACAATTTTAATTGGGTGGAACACAG
CAGGGTAGGATTAGGCAGAATGAGGCAGATTTAGGCAGCGGAAATTTATTTACGCGTC
CATGGCAATTGGAGCTCCTGCAG GCGGCCGC-3′.

### Embodiment 3 Silencing Effect of p26-D4

In this embodiment, GFP gene genetically expressed by Fusarium graminearum PH-1 is used as the target gene to illustrate the silencing effect of p26-D4.

1. Construction of Gene Silencing Vector Based on p26-D4

Based on p26-D4 vector, as for the target gene GFP, VIGS vector containing different fragments of GFP gene is constructed to verify the silencing effect and the size range of exogenous fragment insertion. Eight vectors are constructed: p26-D4-GFP75F, p26-D4-GFP75R, p26-D4-GFP150F, p26-D4-GFP150R, p26-D4-GFP300F, p26-D4-GFP300R, p26-D4-GFP450F and p26-D4-GFP450R. The specific construction method is to amplify GFP gene fragments with different lengths and directions by RCR method, connect the amplified fragments to p26-D4 vector by T4 DNA ligase, and finally screen the target silencing vector.

GFP gene fragments with different lengths and directions are amplified by specific primers, and the specific primers are shown in Table 2.

TABLE 2
Primers for construction of GFP gene
silencing vector based on p26-D4

| Primer name | Primer sequence (5′-3′) |
|---|---|
| p26-D4-GFP75F-F | CCCACCGGTCAGCACGACTTCTTC |
| (SEQ ID NO: 18) | |
| p26-D4-GFP75F-R | CCAATGCATGCCGTCGTCCTTGAAG |
| (SEQ ID NO: 19) | |
| p26-D4-GFP75R-F | CCCACCGGTGCCGTCGTCCTTGAAG |
| (SEQ ID NO: 20) | |
| p26-D4-GFP75R-R | CCAATGCATCAGCACGACTTCTTC |
| (SEQ ID NO: 21) | |
| p26-D4-GFP150F-F | CCCACCGGTATGGTGAGCAAGGGCGAG |
| (SEQ ID NO: 22) | |
| p26-D4-GFP150F-R | CCAATGCATGGTGCAGATGAACTTC |
| (SEQ ID NO: 23) | |
| p26-D4-GFP150R-F | CCCACCGGTGGTGCAGATGAACTTC |
| (SEQ ID NO: 24) | |
| p26-D4-GFP150R-R | CCAATGCATATGGTGAGCAAGGGCGAG |
| (SEQ ID NO: 25) | |
| p26-D4-GFP300F-F | CCCACCGGTCAGCACGACTTCTTCAAG |
| (SEQ ID NO: 26) | |
| p26-D4-GFP300F-R | CCAATGCATGGCGAGCTGCACGCTG |
| (SEQ ID NO: 27) | |
| p26-D4-GFP300R-F | CCCACCGGTGGCGAGCTGCACGCTGC |
| (SEQ ID NO: 28) | |
| p26-D4-GFP300R-R | CCAATGCATCAGCACGACTTCTTCAAG |
| (SEQ ID NO: 29) | |
| p26-D4-GFP450F-F | CCCACCGGTATGGTGAGCAAGGGCGAG |
| (SEQ ID NO: 30) | |
| p26-D4-GFP450F-R | CCAGTTGTGGCTGTTGTAGTTGTAC |
| (SEQ ID NO: 31) | |
| p26-D4-GFP450R-F | CCCACCGGTGTTGTGGCTGTTGTAG |
| (SEQ ID NO: 32) | |
| p26-D4-GFP450R-R | CCAATGCATATGGTGAGCAAGGGCGAG |
| (SEQ ID NO: 33) | |

The protoplast preparation of Fusarium graminearum, PEG-mediated transfection of p26-D4-based gene silencing vector and observation of colony morphology of strains are all the same as those in Embodiment 1.

### 2. Fluorescence Observation

A PDA plate with cellophane is prepared, laid with a sterile cover glass, a 3 mm fresh mycelian block is placed at a distance of 2 cm from the cover glass, and cultured in the dark for 2 days. The fluorescence is observed when the mycelium grows to one third of the cover glass position. The fluorescence intensity is analyzed by ImageJ software.

### 3. Results

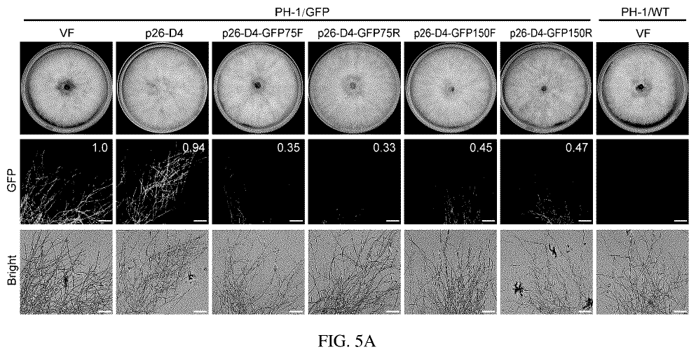

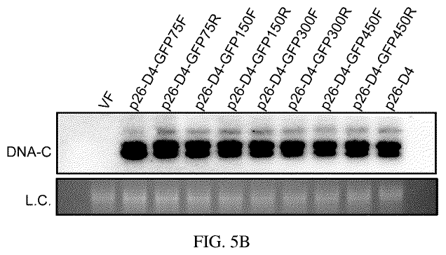

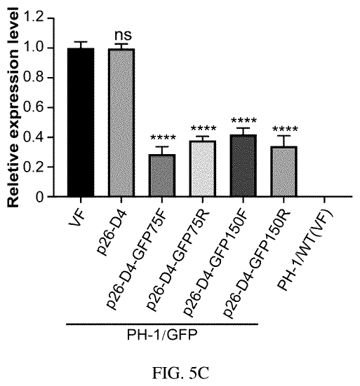

FIG. 5A, FIG. 5B, and FIG. 5C show the silencing efficiency analysis of GFP gene by VIGS vector p26-D4. The results show that the GFP silencing vectors p26-D4-GFP75F, p26-D4-GFP75R, p26-D4-GFP150F, p26-D4-GFP150R, p26-D4-GFP300F, p26-D4-GFP300R, p26-D4-GFP450F and p26-D4-GFP450R constructed based on p26-D4 obtain transfectant through transfection by PEG-mediated protoplasts and re-culture (see FIG. 5B). Southern blot and sequencing analysis confirm that in the strains infected by p26-D4-GFP300F, p26-D4-GFP300R, p26-D4-GFP450F and p26-D4-GFP450R, the inserted GFP fragments of 300 bp and 450 bp are lost in the DNA-C component. However, in the strains infected by p26-D4-GFP75F, p26-D4-GFP75R, p26-D4-GFP150F and p26-D4-GFP150R, the inserted GFP fragments of 75 bp and 150 bp are capable of being stably retained in the viral DNA-C component. The strains infected by p26-D4-GFP75F, p26-D4-GFP75R, p26-D4-GFP150F and p26-D4-GFP150R show normal growth phenotype (see FIG. 5A). The fluorescence intensity is observed by fluorescence microscope, and it is found that the fluorescence intensity of GFP in the strains infected by p26-D4-GFP75F, p26-D4-GFP75R, p26-D4-GFP150F and p26-D4-GFP150R is obviously weaker than the fluorescence intensity of GFP in PH-1/GFP strain and PH-1/GFP strain infected by p26-D4 (see FIG. 5A). Quantitative polymerase chain reaction (qPCR) further proves that compared with PH-1/GFP strain and PH-1/GFP strain infected by p26-D4, the mRNA expression level of GFP in strains infected by p26-D4-GFP75F, p26-D4-GFP75R, p26-D4-GFP150F and p26-D4-GFP150R decrease by 55%-75% (as shown in FIG. 5C). Therefore, the present disclosure confirms that the VIGS silencing vector based on p26-D4 may effectively silence GFP, and the inserted fragment is about 75 bp-150 bp.

### Embodiment 4 Silencing Effect of p26-D4

In this embodiment, the endogenous genes Tri101 and FgPP1 of Fusarium graminearum PH-1 are used as target genes to illustrate the silencing effect of p26-D4.

1. Construction of the Silencing Vectors p26-D4-Tri101 and p26-D4-FgPP1

On the basis of p26-D4 vector, VIGS vectors p26-D4-Tri101 and p26-D4-FgPP1 are constructed for target genes Tri101 and FgPP1. The specific construction method is to amplify 150 bp Tri101 and FgPP1 gene fragments by RCR method, connect the amplified fragments to p26-D4 vector by homologous recombination method, and finally obtain the target silencing vectors by screening.

The 150 bp Tri101 and FgPP1 gene fragments are amplified by specific primers, and the specific primers are shown in Table 3.

TABLE 3
Primers for construction of silencing
vectors p26-D4-Tri101 and p26-D4-FgPP1

| Primer name | Primer sequence (5′-3′) |
|---|---|
| p26-D4-Tri101-F | CAGCACTAACACGTGTACCGGTGATTTGTACT |
| (SEQ ID NO: 34) | CTGTTCCC |
| p26-D4-Tri101-R | GTATGTCCACCCAATCATGCATGGTAGCTGGC |
| (SEQ ID NO: 35) | CGAGGGTGTC |
| p26-D4-FgPP1-F | CAGCACTAACACGTGTACCGGTACCATCTGCT |
| (SEQ ID NO: 36) | TGCTCCTCGCC |
| p26-D4-FgPP1-R | GTATGTCCACCCAATCATGCATGAAAGTCTTC |
| (SEQ ID NO: 37) | CACAACTTG |

The protoplast preparation of Fusarium graminearum PH-1, PEG-mediated transfection of p26-D4-based gene silencing vector and observation of colony morphology of strains are all the same as those in Embodiment 1.

The sequence of the target gene Tri101 is shown
in SEQ ID NO: 3:
GATTTGTACTCTGTTCCCAAGCGTCATCTTTCTCAGCGCAGCACTTCTA
TAATTTAGCGGCCTCACCTTCTGTAACACCAACACCAAGTGATTTACAA
ACACCACCAAAATGGCTTTCAAGATACAGCTCGACACCCTCGGCCAGCT
ACC.
The target gene FgPP1 has a sequence as shown
in SEQ ID NO: 4:
ACCATCTGCTTGCTCCTCGCCTACAAGATCAAGTACCCCGAAAACTTCT
TCATCCTTCGAGGTAACCACGAGTGTGCCTCCATCAACCGTATTTATGG
ATTCTACGACGAGTGCAAGCGTCGCTATAACATCAAGTTGTGGAAGACT
TTC.

### 2. Results

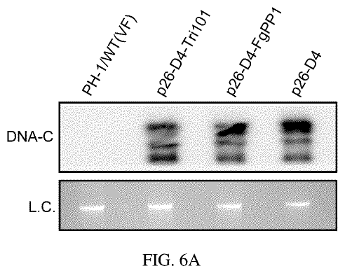

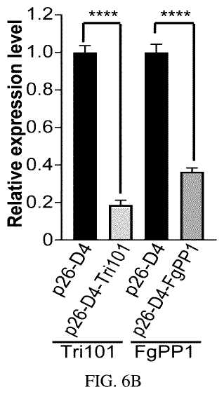

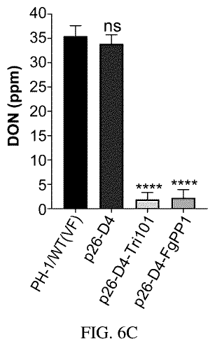

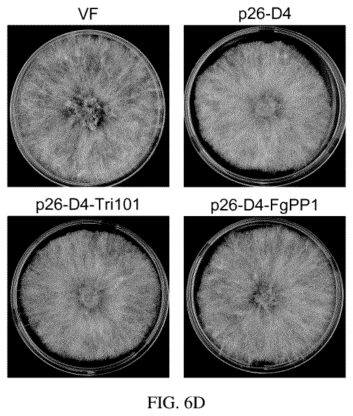

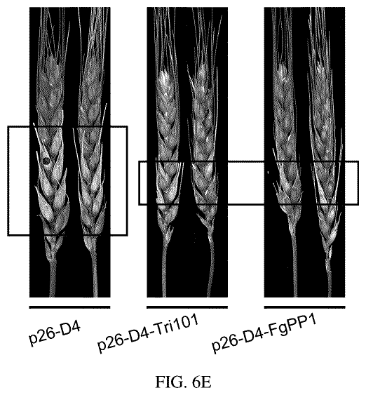

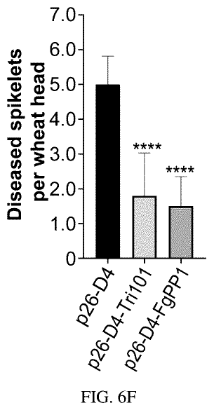

FIG. 6A, FIG. 6B, FIG. 6C, FIG. 6D, FIG. 6E, and FIG. 6F show an analysis of silencing efficiency of endogenous genes Tri101 and FgPP1 of Fusarium graminearum. The results show that the silencing vectors p26-D4-Tri 101 and p26-D4-FgPP1 constructed based on p26-D4 obtain the transfectant by transfection of PEG-mediated protoplast and re-culture. Southern blot analysis confirms that the virus components could be effectively replicated in strains infected by p26-D4-Tri101 and p26-D4-FgPP1 (see FIG. 6A). Compared with strains infected by p26-D4, the RNA expression of the gene Tri101 in strains infected by p26-D4-Tri101 decreases by 80%, and the RNA expression of FgPP1 in strains infected by p26-D4-FgPP1 decreases by 63% (see FIG. 6B). The phenotypes of the strains infected by p26-D4-Tri101 and p26-D4-FgPP1 are not obviously abnormal, which are consistent with wild-type strain PH-1 and strains infected by p26-D4 (see FIG. 6D). In addition, the experiments of toxin production detection and pathogenicity test show that compared with strains infected by p26-D4, the DON production and pathogenicity of strains infected by p26-D4-Tri101 and p26-D4-FgPP1 are greatly reduced (see FIG. 6C, FIG. 6E and FIG. 6F). It shows that p26-D4 constructed by the disclosure is capable of effectively silencing the endogenous genes of Fusarium graminearum, and at the same time, it shows that the VIGS vector is capable of successfully transforming pathogenic fungal strains into hypovirulent strains.

### Embodiment 5 Analysis of Biological Control Effect of VIGS-Induced Hypovirulent Strains

Based on the results of Embodiment 4, the biological control effect of obtained hypovirulent strains infected by p26-D4-Tri101 and p26-D4-FgPP1 is further tested. In this embodiment, co-infection method and pre-spray method are selected to verify the biological control effect of VIGS-induced hypovirulent strains.

Co-infection method (Test 1): the mycelian blocks of the hypovirulent strains infected by p26-D4-Tri101 and p26-D4-FgPP1 are cut into small blocks with the same size and inoculated into the same spikelet together with 10 μL of PH-1 conidia suspension. After 12 days, the incidence of wheat Fusarium head blight (FHB) is observed, the number of diseased spikelets is counted and the content of deoxynivalenol (DON) is determined.

Pre-spray method (Test 2): the hypovirulent strains infected by p26-D4-Tri101 and p26-D4-FgPP1 are cultured in PDB liquid medium for 3 days, and the mycelium is collected, broken into small mycelium segments, and the OD₆₀₀≈2.0. First, the mycelium segment suspension is sprayed on wheat spikelets, and then 10 μL of PH-1 conidia suspension is inoculated after 24 h. After 12 days, the incidence of wheat FHB is observed, the number of diseased spikelets is counted and the DON content is determined.

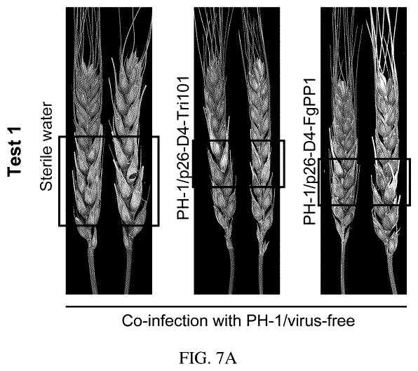

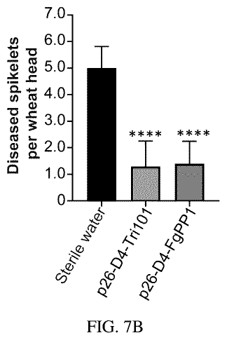

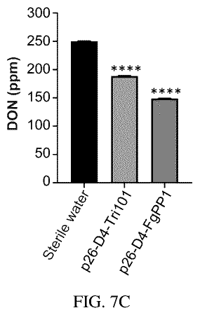

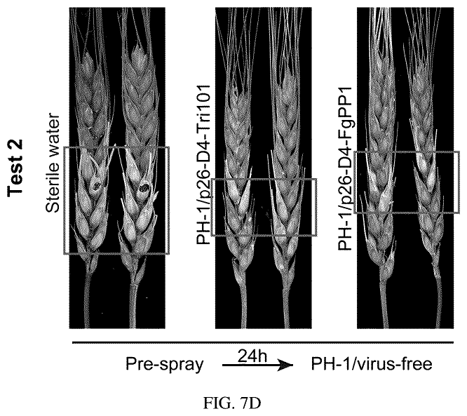

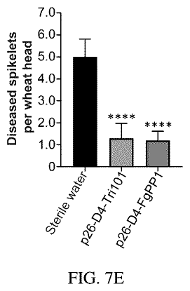

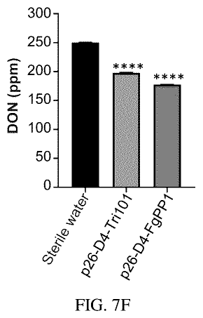

FIG. 7A, FIG. 7B, FIG. 7C, FIG. 7D, FIG. 7E, and FIG. 7F illustrate an analysis on the control effect of VIGS-induced hypovirulent strains on wheat Fusarium head blight (FHB). The results show that in the co-infection method (Test 1), compared with the control group treated with sterile water, the wheat spikelets treated with hypovirulent strains infected by p26-D4-Tri101 and p26-D4-FgPP1 show obviously delayed expansion of wheat FHB spot, the protection efficiency reaches 74% and 76% respectively, and the DON content decreases by 21% and 29% respectively (see FIG. 7A, FIG. 7B, FIG. 7C). In the determination test by pre-spray method (Test 2), compared with the control group treated with sterile water, the wheat spikelets treated with hypovirulent strains infected by p26-D4-Tri101 and p26-D4-FgPP1 also show obviously delayed expansion of wheat FHB spot, the protection efficiency reaches 72% and 74% respectively, and the DON content decreases by 25% and 40% respectively (see FIG. 7D, FIG. 7E, FIG. 7F). The results show that the low-virulence strain induced by VIGS vector p26-D4 could effectively control wheat FHB under experimental conditions.

The above-mentioned embodiments only describe the preferred mode of the disclosure, and do not limit the scope of the disclosure. Under the premise of not departing from the design spirit of the disclosure, various modifications and improvements made by ordinary technicians in the field to the technical scheme of the disclosure shall fall within the protection scope determined by the claims of the disclosure.

1. A mycovirus-induced gene silencing vector, wherein its nucleotide sequence is shown in SEQ ID NO: 2.

2. A construction method for the mycovirus-induced gene silencing vector according to claim 1, comprising following steps:
(1) connecting three single-stranded circular DNA molecules DNA-A as shown in SEQ ID NO: 5, DNA-B as shown in SEQ ID NO: 6 and DNA-C as shown in SEQ ID NO.: 7 of mycovirus FgGMTV1/HB58 in series and introducing into a same vector to construct a recombinant vector; and
(2) carrying out a deletion mutation on a coding protein p26 of the DNA-C molecule in the recombinant vector to obtain the mycovirus-induced gene silencing vector.

3. The construction method according to claim 2, wherein 1.3 copies of the DNA-A,1.3 copies of the DNA-B and 1.5 copies of the DNA-C are connected in series, and then connected to pBluescript II SK(+) to construct the recombinant vector.

4. (canceled)

5. The construction method according to claim 2, wherein the deletion mutation is a deletion of a sequence of 454-603 nt of the coding protein p26 of the DNA-C as shown in SEQ ID NO.: 7.

6. The mycovirus-induced gene silencing vector according to claim 1, wherein the mycovirus-induced gene silencing vector is contained in Fusarium graminearum.

7. The mycovirus-induced gene silencing vector according to claim 6, wherein the mycovirus-induced gene silencing vector carries exogenous genes, and the exogenous genes comprise Tri101 gene and FgPP1 gene.
